(19) United States
(12) Patent Application Publication
(10) Pub. No.: US 2012/0251571 A1
(43) Pub. Date: Oct. 4, 2012

(54) RNASE L-MEDIATED CLEAVAGE PRODUCTS AND USES THEREOF

(75) Inventors: Robert H. Silverman, Beachwood, OH (US); Malathi Krishnamurthy, Ypsilanti, MI (US)

(73) Assignee: The Cleveland Clinic Foundation, Cleveland, OH (US)

(21) Appl. No.: 13/247,961

(22) Filed: Sep. 28, 2011

Related U.S. Application Data
(60) Provisional application No. 61/387,254, filed on Sep. 28, 2010.

(51) Int. Cl. A61K 31/7088; A61P 37/04; A61P 31/14; C07H 21/02; C12N 5/071

(52) U.S. Cl. 424/228.1; 536/24.5; 435/375; 435/366

(57) ABSTRACT

The invention is directed to one or more RNase L mediated cleavage products. In particular aspects, the RNase L mediated cleavage products are RNase L mediated cleavage products of a virus, referred to herein as a “suppressor of virus ribonucleic acid (RNA)” or “svRNA” and uses thereof.

### RELATED APPLICATION(S)

This application claims the benefit of U.S. Provisional Application No. 61/387,254, filed on Sep. 28, 2010. The entire teachings of the above application are incorporated herein by reference.

### GOVERNMENT SUPPORT

This invention was made with government support under 1RC1A1086041, CA44059, DA024563, and AI060389 awarded by the National Institutes of Health. The government has certain rights in the invention.

### BACKGROUND OF THE INVENTION

Triggering and propagating an intracellular innate immune response is essential for control of viral infections. RNase L is a host endoribonuclease and a pivotal component of innate immunity that cleaves viral and cellular RNA within single-stranded loops releasing small structured RNAs with 5′-hydroxyl (5′-OH) and 3′-monophosphoryl (3′-p) groups. In 2007, it was reported that RNase L cleaves self RNA to produce small RNAs that function as pathogen-associated molecular patterns (PAMPs) (Silverman, R., et al., J Virol., 81:12720-12729 (2007)). However, the precise sequence and structure of PAMP RNAs produced by RNase L is unknown.

A greater understanding of the role of RNase L in innate immunity is needed.

### SUMMARY OF THE INVENTION

Described herein is the use of hepatitis C virus RNA as substrate to characterize RNase L mediated cleavage products (referred to herein as suppressor of virus RNA (svRNA)) for their ability to activate RIG-I like receptors (RLR). The NS5B region of HCV RNA was cleaved by RNase L to release an svRNA that bound to RIG-I, displacing its repressor domain and stimulating its ATPase activity while signaling to the IFN-β gene in intact cells. All three of these RIG-I functions were dependent on the presence in svRNA of the 3′-p. Furthermore, svRNA suppressed HCV replication in vitro through a mechanism involving IFN production and triggered a RIG-I-dependent hepatic innate immune response in mice. RNase L and OAS (required for its activation) were both expressed in hepatocytes from HCV-infected patients, raising the possibility that the OAS/RNase L pathway might suppress HCV replication in vivo. Shown herein is that RNase L mediated cleavage of HCV RNA generates svRNA that activates RIG-I, thus propagating innate immune signaling to the IFN-β gene.

Accordingly, in one aspect, the invention is directed to an (one or more) isolated nucleic acid sequence comprising an svRNA (e.g., an HCS svRNA). In a particular aspect, the invention is directed to an isolated sequence comprising SEQ ID NO: 1. In other aspects, the invention is directed to an isolated nucleic acid sequence comprising SEQ ID NO: 3; an isolated nucleic acid sequence comprising SEQ ID NO: 4; an isolated nucleic acid sequence comprising SEQ ID NO: 25; an isolated nucleic acid sequence comprising SEQ ID NO: 26; and an isolated nucleic acid sequence comprising SEQ ID NO: 27.

The sequence can further comprise one or more hydroxyl (—OH) groups, one or more monophosphoryl (-p) groups, one or more single stranded overhangs or a combination thereof. For example the sequence can further comprise a 5′-OH and a 3′-p; a 5′-p3 and a 3′-OH; a 5′ single stranded overhang, a 3′ single stranded overhang; or combinations thereof.

In another aspect, the invention is directed to a pharmaceutical composition comprising an (one or more) svRNA sequence (e.g., SEQ ID NO: 1; SEQ ID NO: 3; SEQ ID NO: 4; SEQ ID NO: 25; SEQ ID NO: 26; SEQ ID NO: 27).

In another aspect, the invention is directed to a method of inducing an immune response to a hepatitis C virus (HCV) in a cell comprising introducing into the cell a composition comprising a HCV svRNA; and maintaining the cell under conditions in which the svRNA stimulates RIG-1 signaling and propagates signaling to the IFN-β gene, thereby inducing an immune to the HCV in the cell.

In another aspect, the invention is directed to a method of inducing an immune response to HCV in an individual in need thereof comprising administering a therapeutically effective amount of a composition comprising a HCV svRNA that stimulates RIG-1 signaling and propagates signaling to the IFN-β gene, thereby inducing an immune response to the HCV in the individual.

In another aspect, the invention is directed to a method of treating a HCV in an individual in need thereof comprising administering a therapeutically effective amount of a composition comprising a HCV svRNA (e.g., HCV svRNA3) that stimulates RIG-1 signaling and propagates signaling to the IFN-β gene, thereby inducing an immune response to the HCV in the individual.

### BRIEF DESCRIPTION OF THE DRAWINGS

FIGS. 1A-1C show cleavage of HCV RNA by RNase L produces small RNAs with PAMP activity. (1A) Activation of the IFN-β promoter after 18 h in Huh7 cells in response to transfection with undigested or RNase L-digested HCV RNA segments. (1B) RNase L releases svRNA3 from two adjacent stem-loops in the NS5B region of the HCV open reading frame. (1C) Detection of RNase L-mediated cleavage product of HCV RNA (svRNA3) in Huh7.5 cells at 96 h after electroporated with full-length HCV genomic RNA or a region of HCV RNA encoding svRNA3 (nt 8703-9416) as determined in a Northern blot (upper) (Materials and Methods). Small RNAs, <200 nt (10 μg), and 100 ng of svRNA3 were stained with gelstar (Cambrex Bio Science) (lower).

FIGS. 2A-2J show activation of RIG-I ATPase activity and IFN-β induction by svRNA3 and its derivatives. Predicted RNA secondary structure of (2A) svRNA3 (5′-p₃/3′-OH); (2B) svRNA3 (5′-OH/3′-p); (2C) CsvRNA3 (5′-p₃/3′-OH), complement of svRNA3; (2D) svRNA3 short (5′-OH/3′-OH or 5′-OH/3′-p), the main stem-loop structure of svRNA3; (2E) 5′ΔsvRNA3 (5′-p₃/3′-OH), svRNA3 deleted for the 5′-ss overhang; (2F) 3′ΔsvRNA3 (5′-p₃/3′-OH), svRNA3 deleted for the 3′-ss overhang; (2G) 5′ 3′ΔsvRNA3 (5′-p₃/3′-OH), svRNA3 deleted for both the 3′- and 5′-ss overhangs. (2H) Activation of RIG-I or MDA5 ATPase (20 min at 37° C.) by the indicated RNAs. (2I) Activation of the IFN-β promoter in Huh7 cells after 18 h by transfection of the indicated RNAs. (2J) Induction of IFN-β by svRNA3 in mouse embryo fibroblasts (met). IFN-β levels in supernatants of WT, RIG-I-deficient, MDA5-deficient, or IPS1-deficient mouse embryo fibroblasts 18 h after transfection with 30 pmol of svRNA3. IFN-β protein levels were measured by ELISA. RNA structures were as predicted by mfold software (Zuker 2003).

FIGS. 3A-3B show svRNA3 binds RIG-I causing the release of its repressor domain (RD). (3A) RIG-I binding of svRNA3 with different termini (as shown) as determined by gel-shift analysis of full-length (FL) RIG-I or its N-terminal (N) polypeptide (1-228 amino acids). (3B) Release of RIG-I RD by partial trypsin digestion. The silver-stained gel image shows trypsin digestion products of Flag-RIG-I incubated with 3, 6, 15, or 30 pmol of the indicated RNA.

FIGS. 4A-4F show HCV svRNA3 induces a hepatic innate immune response and suppresses HCV replication by a paracrine mechanism. (4A-4E) Hydrodynamic tail vein injection of poly-U/UC (5′-p₃/3′-OH) or svRNA3 (5′-OH/3′-p) in WT and Rig-I^{-/-} mice (n=3). (4A) Induction of circulating IFN-β by poly-U/UC or svRNA3. (4B) Hepatic induction of ISG54 protein as determined by immunohistochemistry (IBC) by poly-U/UC or svRNA3. (4C-4E) Hepatic induction of Isg56, Ifnb, and Rig-i mRNAs by poly-U/UC or svRNA3 as determined by qRT-PCR. (F) Paracrine inhibition of HCV infections by poly-U/UC (5′-p₃/3′-OH) or svRNA3 (5′-OH/3′-p). Huh7.5 cells (in triplicate) were treated for 12 h before HCV infection with medium containing IFN-β or conditioned medium from Huh7 cells transfected with the indicated RNAs. The graph shows the number of infected cells as determined by focus-forming unit (FFU) assay at 48 h after infection.

FIGS. 5A-5B shows inhibition of HCV replication in response to 2-5A activation of RNase L. (5A) RNase L levels in lysates (30 μg) of DU145 prostate cancer cells and hepatoma cell lines Huh7 and Huh7.5 as determined in immunoblots (different exposures of the same blot are shown). Blots were probed with monoclonal antibody reactive to human RNase L (Dong and Silverman 1995) and compared to anti-β-actin as control. (5B) Huh7 cells were either mock transfected or transfected with 2-5A trimer (p₃5′A2′p5′A2′p5′A) before or after HCV infection (JFH1 strain) for 8 h. Supernatants were harvested at 48 h post-infection and titered for HCV by focus-forming assays.

FIGS. 6A-6B show hepatic expression of HCV NS5A, OAS1, and RNase L in HCV-infected liver. Images show a 0.15 μm optical section of liver biopsy specimen stained with Draq-5 to show nuclei (blue), and immunostained with antibodies specific to NS5A (red) and (6A) OAS1 (green) or (6B) RNase-L (green). The left panel of each set features the merged image showing sites of protein codistribution (yellow). Images represent a single patient and are representative of analyses from six different patients with chronic HCV infection (data not shown). 60× magnification.

FIG. 7 shows hypothetical temporal appearance and roles of the PAMPs, poly-U/UC, and svRNA3, in the innate immune response against HCV infections. Active (A) and inactive (I) forms of OAS and RNase L are indicated.

FIG. 8 is a schematic of the SvRNA cloning strategy. HCV RNA segments were cleaved in vitro by RNase L activated with 2-5A. The isolated pool of <200 nt RNAs were incubated with either Flag-RIG-1 or Flag-MDA5 and the immobilized RNAs were cloned and sequenced.

FIGS. 9A-9C show expression of RNase L and HCV proteases in Huh7.5 cells electroporated with HCV RNA. (9A) Expression of flag-RNase L at 48 post-transfection as determined by immunoblotting with anti-flag. (9B) Expression of HCV core protein in Huh7.5 cells 48 h and 96 h after electroporation of full-length HCV genomic RNA determined in an immunoblot. (9C) Activation of RNase L in intact Huh7.5 cells after 2-5A or poly(rl):poly (rC) treatment as determined by monitoring rRNA cleavage products (arrows) at 96 h after electroporation with HCV RNA (see Materials and Methods).

FIGS. 10A-10B show induction of IFN-β by svRNA2 in mouse embryo fibroblasts (mef). IFN-β levels in supernatants of WT, RIG-1-deficient, MDA5-deficient or IPS1-deficient mouse embryo fibroblasts 18 h after transfection with (10A) 30 pmol of svRNA1, and (10B) svRNA2, both produced by chemical synthesis. IFN-β protein levels were measured by ELISA.

### DETAILED DESCRIPTION OF THE INVENTION

Viral RNAs, often in the form of cytoplasmic 5′-triphosphorylated, double-stranded, or uridine and adenosine-rich viral RNAs, are pathogen-associated molecular patterns (PAMPs) that trigger innate immunity through RIG-I-like receptors (RLR), a family of cytoplasmic pathogen recognition receptors (PRRs) (Horner and Gale 2009; Rehwinkel and Reis e Sousa 2010; Ting et al. 2010; Yoneyama and Fujita 2010). These RNA PAMPs interact with either of two RLRs, RIG-I and MDA5, containing N-terminal caspase activation and recruitment domains (CARD) and C-terminal DExD/H Box RNA helicase motifs (Yoneyama et al. 2004; Kato et al. 2005, 2006; Gitlin et al. 2006). Subsequently, RIG-I and MDA5 interact with another CARD protein, IPS-1 (MAVS, VISA, Cardif), in the mitochondrial membrane (Kawai et al. 2005; Meylan et al. 2005; Seth et al. 2005; Xu et al. 2005; Loo et al. 2006). IPS-1 then relays the signal to the kinases, IKKε and TBK1 that phosphorylate transcription factor IRF-3. Transcription factor NF-κB is simultaneously activated through IPS-1. The homodimerized and phosphorylated IRF-3 relocalizes to the nucleus along with activated NF-κB and independently or together activate different target genes, including the IFN-β gene.

The IFN response against RNA viruses is frequently mediated by RNase L, part of a ribonucleolytic pathway containing the PRR, 2′-5′-oligoadenylate synthetase (OAS) (Silverman 2007). Type I IFNs induce at the transcriptional level a group of OAS proteins that are activated by viral dsRNA PAMPs to produce 2-5A [pₓ5′A(2′p5′A)ₙ; x=1-3; n≧2] from ATP (Hovanessian et al. 1977; Kerr and Brown 1978; Hovanessian and Justesen 2007). OAS activators include viral replicative intermediates, ds RNA genomes, annealed ss RNAs of opposite polarity and highly structured ss RNA. 2-5A is the ligand and activator of RNase L, a ubiquitous enzyme in mammalian cells, including primary human hepatocytes, that lies dormant until viral infections occur (Zhou et al. 2005). Human RNase L is a 741 amino acid polypeptide containing, from N- to C-termini, nine ankyrin repeats, several protein kinase-like motifs, and the ribonuclease domain (Hassel et al. 1993; Zhou et al. 1993). 2-5A binds to ankyrin repeats 2 and 4 (Tanaka et al. 2004), causing catalytically inactive RNase L monomers to form activated dimers with potent ribonuclease activity (Dong and Silverman 1995; Cole et al. 1996). Once activated, RNase L cleaves single-stranded regions of viral and host RNAs, principally at UpAp and UpUp dinucleotides, leaving 3′-phosphoryl and 5′-hydroxyl groups at the termini of the RNA cleavage products (Floyd-Smith et al. 1981; Wreschner et al. 1981). Interestingly, cleavage of cellular (self) RNA by RNase L results in the production of short RNAs that activate RNA helicases, RIG-I and MDA5, and the adapter IPS-1 resulting in activation of the IFN-β gene (Malathi et al. 2005, 2007). As a result, circulating levels of IFN-β production were reduced several fold in Sendai virus- or encephalomyocarditis virus-infected RNase L^{-/-} mice compared with infected wild-type mice. In addition, 2-5A treatment of wild-type mice, but not of RNase L^{-/-} resulted in IFN-β production. However, the sequence and structure of the small RNAs produced by RNase L, their interactions with RIG-I and/or MDA5, and their role in mediating antiviral innate immunity have remained largely unexplored.

Hepatitis C virus (HCV), Hepacivirus genus of the Flaviviridae family, is a virus that has infected about four million adults in the United States and is a major cause of chronic hepatitis, liver cirrhosis, and hepatocellular carcinoma (Armstrong et al. 2006). During HCV infections, the viral PAMP that triggers type I IFN production is the polyuridine tract (poly-U/UC) in the 3′ nontranslated region of the viral genomic RNA (Saito et al. 2008). Poly-U/UC requires a 5′-triphosphate to activate RIG-I in the cytoplasm of infected cells. Described herein is the use HCV genomic RNA as a model substrate to characterize the requirements for RLR signaling by RNase L cleavage products. The findings herein demonstrate a requirement for the 3′-monophosphate and complex features in the RNA cleavage product responsible for potent PAMP activity (Malathi, K., et al., RNA, 16(11)::2108-2119 (Nov. 16, 2010) which is incorporated herein by reference in its entirety).

Accordingly, in one aspect, the invention is directed to one or more RNase L mediated cleavage products. In particular aspects, the RNase L mediated cleavage products are RNase L mediated cleavage products of a virus, referred to herein as a “suppressor of virus ribonucleic acid (RNA)” or “svRNA”. Viruses that can be cleaved to generate an svRNA include a deoxyribonucleic acid (DNA) virus or a ribonucleic acid (RNA) virus. In a particular aspect, the virus is a ribonucleic acid (RNA) virus.

Such viruses include hepatitis virus (e.g., hepatitis A virus, hepatitis C virus), human immunodeficiency virus (HIV), xenotropic murine leukemia virus related virus (XMRV), and the like. In one aspect, the virus is hepatitis C virus (HCV). In particular aspects, the HCV is a genotype 1 (e.g., 1a, 1b) HCV, a genotype 2 (e.g., 2a, 2b, 2c) HCV, a genotype 3 (e.g., 3a) HCV, a genotype 4 (e.g., 4a, 4c) HCV, a genotype 5 (e.g., 5a) HCV, a genotype 6 (e.g., 6a) HCV, a genotype 7 (e.g., 7a, 7b) HCV, a genotype 8 (e.g., 8a) HCV, a genotype 9 (e.g., 9a) HCV, a genotype 10 (e.g., 10a) HCV, a genotype 11 (e.g., 11a) HCV, or combinations thereof.

The svRNA can activate retinoic acid-inducible protein (RIG-I) like receptors (RLRs). In a particular aspect, the svRNA is a highly structured RNA that binds to RIG-I, displaces its repressor domain and/or stimulates its ATPase activity while signaling to the IFN-β gene. In more particular aspects, the svRNA comprises a broken-stem-loop with 5′ and 3′ overhangs. In other aspects, the svRNA comprises a 5′ or 3′ hydroxyl group. In yet other aspects, the svRNA comprises a 5′ or 3′ phosphate group. In other aspects, the svRNA comprises one or more single stranded (ss) regions or overhangs.

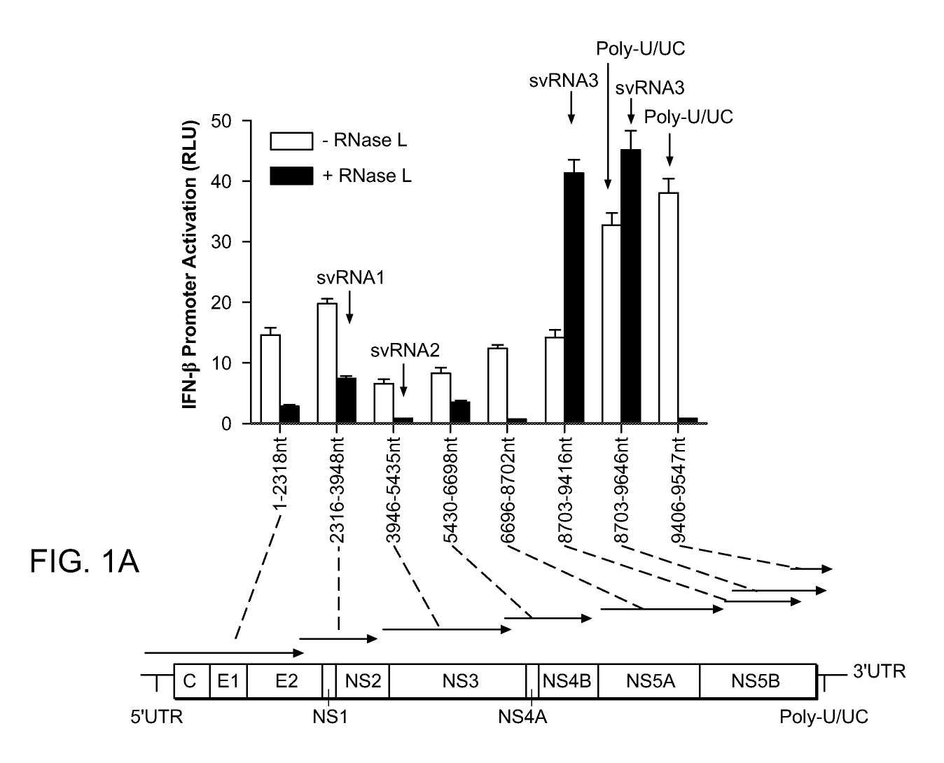

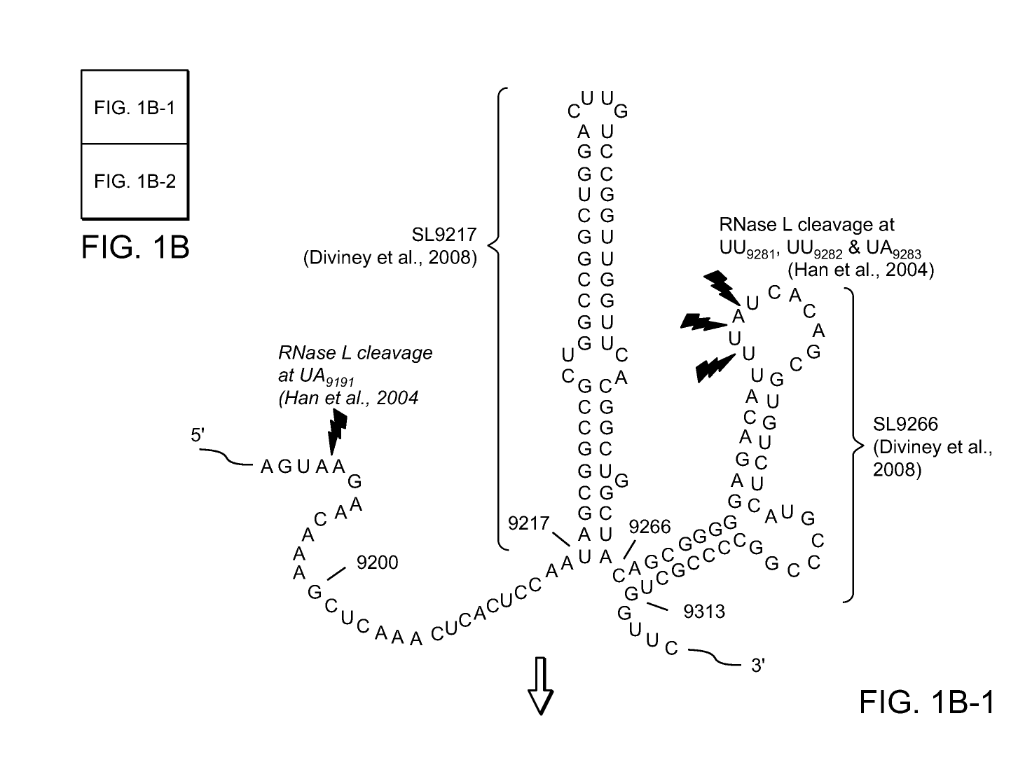

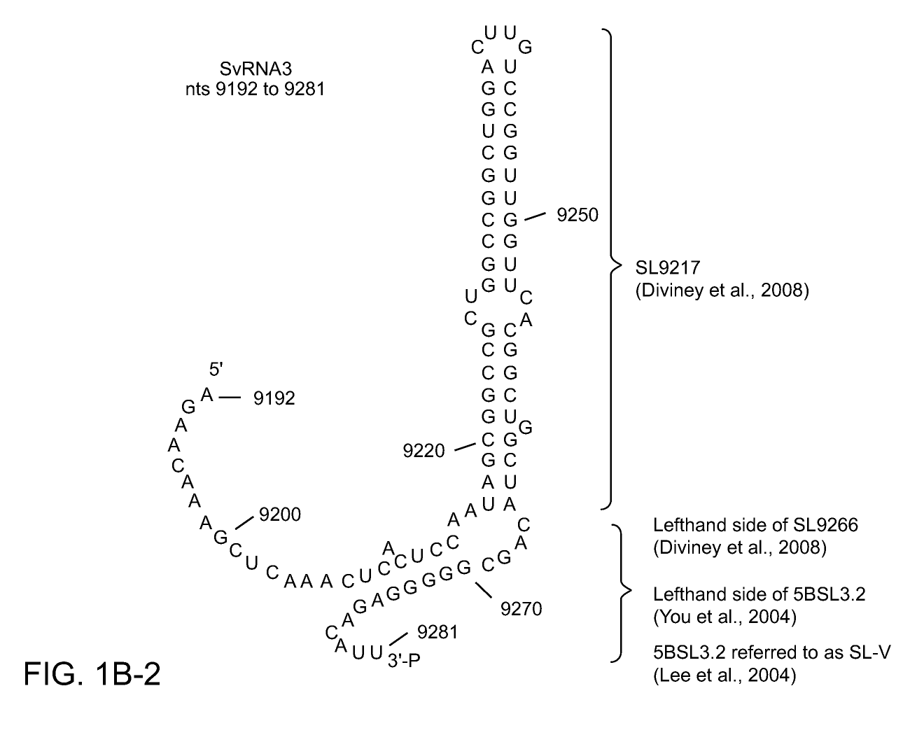

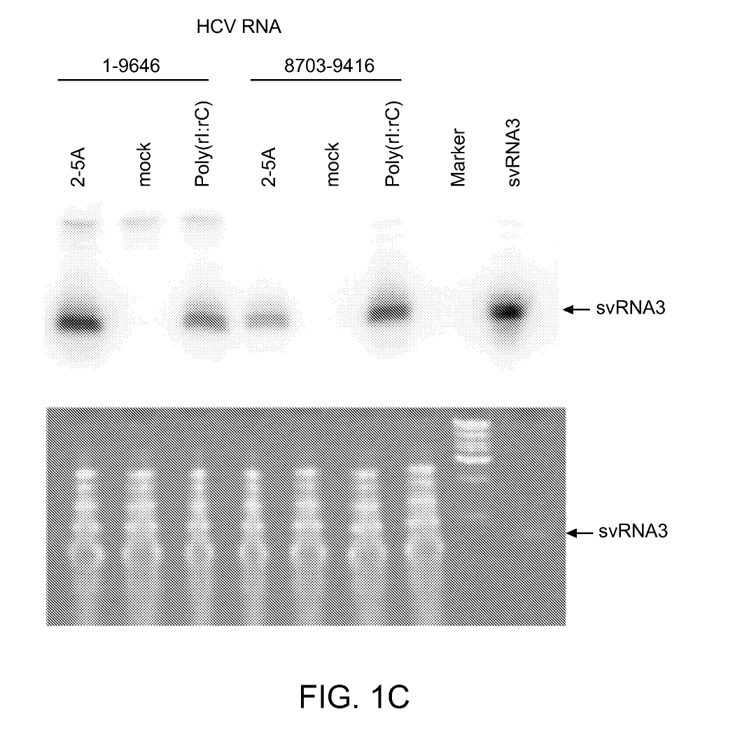

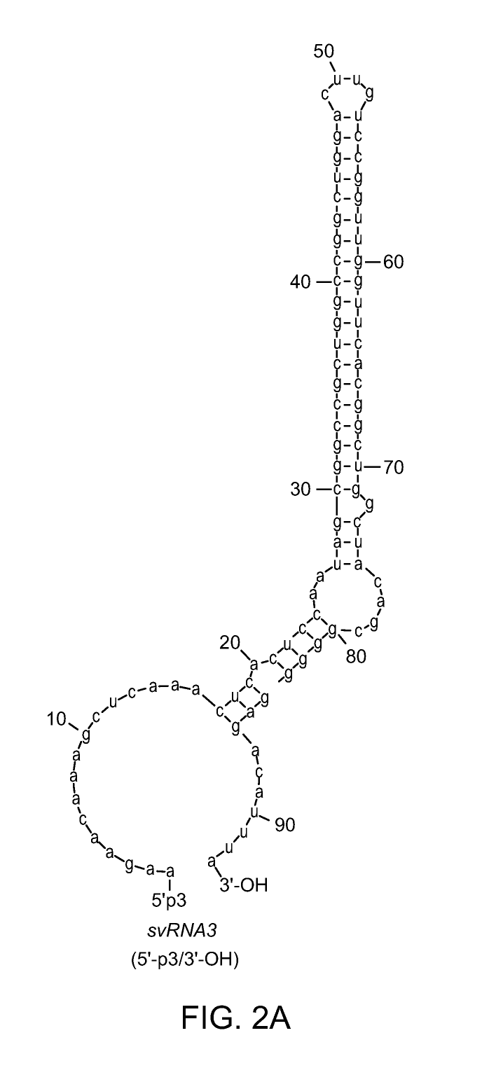

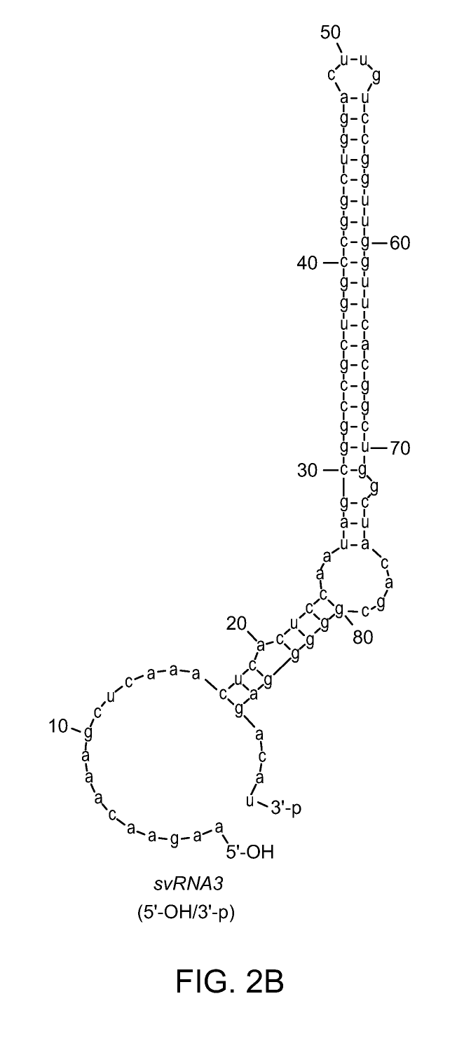

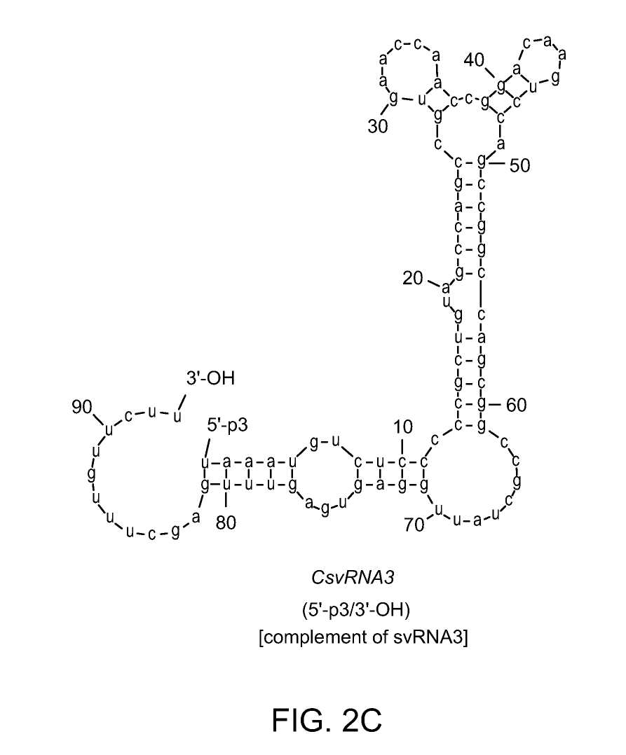

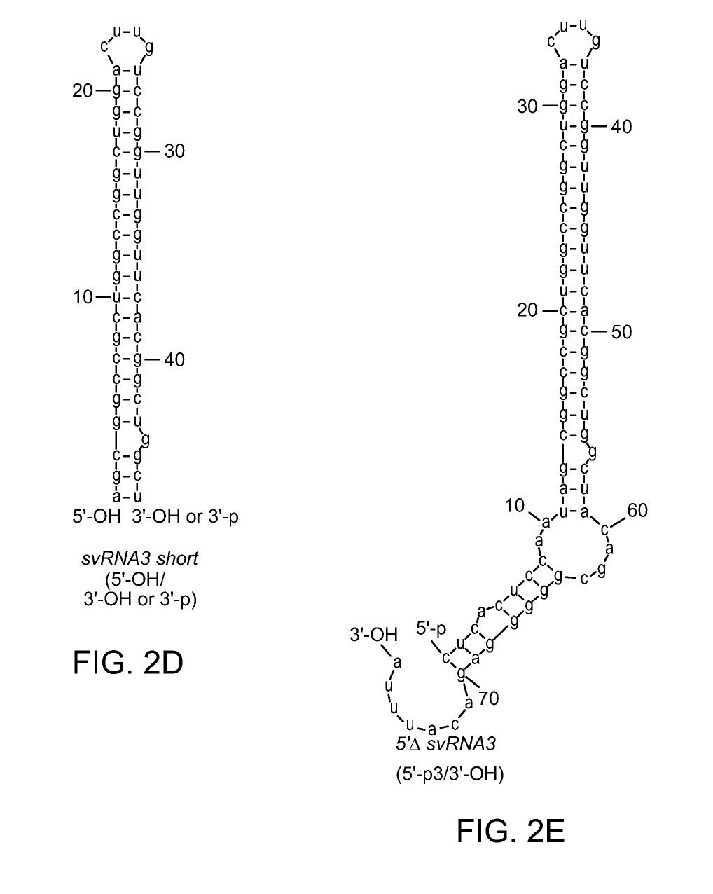

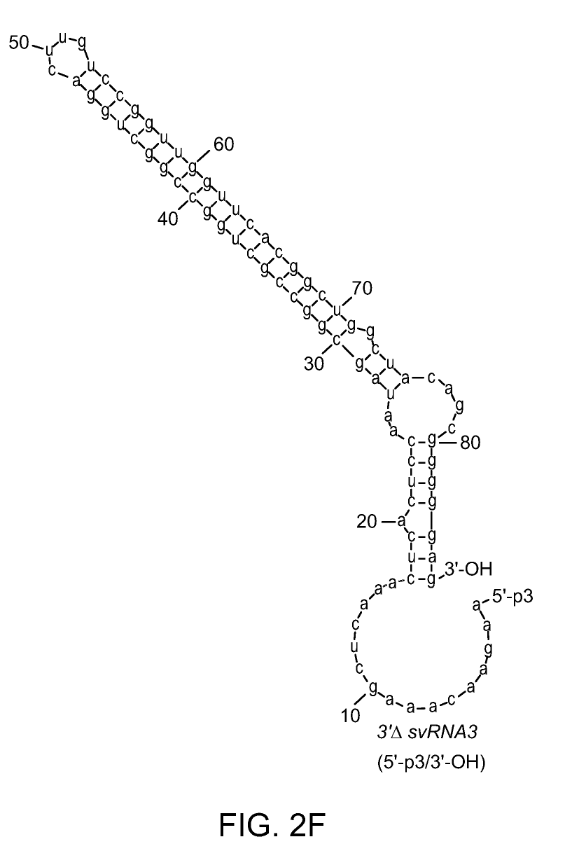

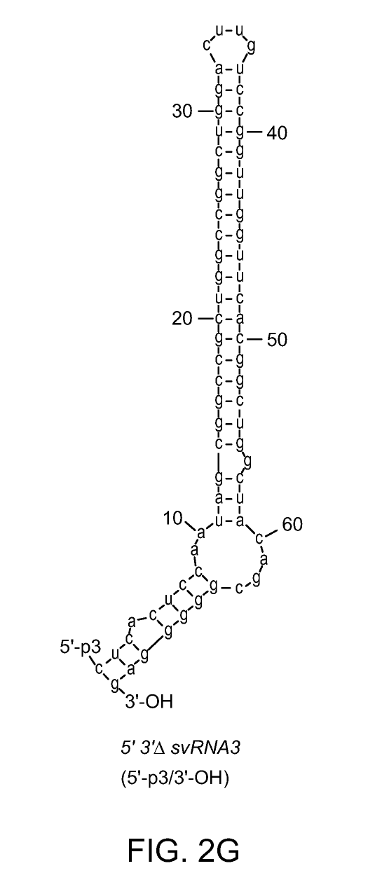

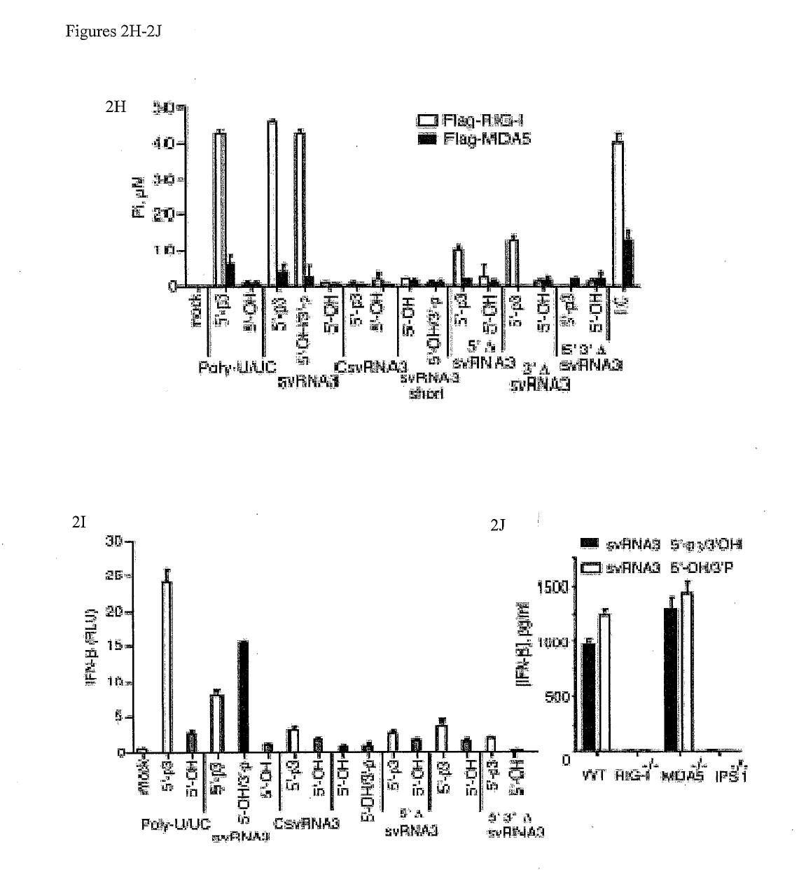

In a particular aspect, the invention is directed to an RNase L mediated cleavage product of a hepatitis C virus (HCV) that activates RLRs (HCV svRNA). For example, the RNase L mediated cleavage product of HCV comprises all or a biologically active portion of the sequence of svRNA3 (or svRNA1, svRNA2) shown in FIGS. 1B, 2A and Table 2. As used herein, a “biologically active” portion is a portion of a svRNA sequence that activate retinoic acid-inducible protein (RIG-I) like receptors (RLRs). In particular aspects, all or a biologically active portion of the HCV svRNA binds to RIG-I, displaces its repressor domain and/or stimulates its ATPase activity while signaling to the IFN-β gene.

In more particular aspects, the invention is directed to an isolated nucleic acid sequence comprising an svRNA. In one aspect, the invention is directed to an isolated nucleic acid sequence comprising SEQ ID NO: 1, SEQ ID NO: 3, SEQ ID NO: 4, SEQ ID NO: 25, SEQ ID NO: 26, or SEQ ID NO: 27. In other aspects, the invention is directed to an isolated sequence that has about 80%, 81%, 82%, 83%, 84%, 85%, 86%, 97%, 88%, 89%, 90%, 91%, 92%, 93%, 94%, 95%, 96%, 97%, 98%, or 99% sequence similarity or identity to SEQ ID NO: 1, SEQ ID NO: 3, SEQ ID NO: 4, SEQ ID NO: 25, SEQ ID NO: 26, or SEQ ID NO: 27.

As used herein “isolated”, “substantially pure,” or “substantially pure and isolated” refers to a nucleic acid molecule or sequence that is separated from nucleic acids that normally flank the nucleotide sequence and/or has been completely or partially purified from other sequences. For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system, or reagent mix. In other circumstances, the material may be purified to essential homogeneity, for example, as determined by agarose gel electrophoresis or column chromatography such as HPLC. Preferably, an isolated nucleic acid molecule comprises at least about 50%, 80%, 90%, 95%, 98% or 99% (on a molar basis) of all macromolecular species present.

In yet other aspects, the isolated nucleic acid comprises one or more hydroxyl (OH) groups, phosphate (p) groups (e.g., monophosphoryl groups), single stranded (ss) regions (overhang regions) or combinations thereof. The hydroxyl group, the phosphate group, and/or the ss overhang can occur at the 5′ and/or the 3′ end of the svRNA. For example, the svRNA can comprise a 5′-OH and a 3′-p; a 5′-p3 and a 3′-OH; a 5′ single stranded overhang, a 3′ single stranded overhang or a combination thereof (e.g., 5′ ss overhang; 3′ss overhang; 5′ ss overhang and a 3′ ss overhang).

As will be appreciated by those of skill in the art, an svRNA can be obtained using recombinant methods or chemically synthesized as described herein.

As shown herein, the svRNA activates RIG-I, thus propagating immune signaling to the IFN-β gene, thereby directing immune signaling against the virus in vitro and in vivo. Thus, the svRNAs described herein can be used as prophylactic agents or as therapeutic agents.

Accordingly, in one aspect, the invention is directed to methods of inducing an immune response to a virus in a cell (e.g., in vitro; in vivo) comprising introducing into the cell a composition comprising a (one or more) svRNA of the virus; and maintaining the cell under conditions in which the svRNA stimulates RIG-1 signaling and propagates signaling to the IFN-β gene, thereby inducing an immune response to the virus in the cell. As used herein an “svRNA of a virus” is an svRNA that is a cleavage product from the cleavage of the virus (e.g., HCV) with RNase L, stimulates RIG-1 signaling and propagates signaling to the IFN-β gene, and thereby induces an immune response to the virus.

In a particular aspect, the invention is directed to a method of inducing an immune response to a hepatitis C virus (HCV) in a cell comprising introducing into the cell a composition comprising a HCV svRNA; and maintaining the cell under conditions in which the svRNA stimulates RIG-1 signaling and propagates signaling to the IFN-β gene, thereby inducing an immune to the HCV in the cell. In particular aspects, the composition comprises SEQ ID NO: 1, SEQ ID NO: 3, SEQ ID NO: 4, SEQ ID NO: 25, SEQ ID NO: 26, SEQ ID NO: 27 or combinations thereof.

As used herein a (one or more) “cell” refers to a cell of an animal, and in a particular aspect, a mammalian cell. Examples of mammalian cells include cells from primates, a canine, a feline, a rodent, and the like. Specific examples include cells of humans, dogs, cats, horses, cows, sheep, goats, rabbits, guinea pigs, rats and mice.

In another aspect, the invention is directed to a method of inducing an immune response to a virus in an individual in need thereof comprising administering a therapeutically effective amount of a composition comprising a (one or more) svRNA of the virus that stimulates RIG-1 signaling and propagates signaling to the IFN-β gene, thereby inducing an immune response to the virus in the individual. The individual can be, for example, an individual who has not been exposed to the virus, an individual who is at risk of exposure to the virus, or an individual who has been exposed to the virus.

In a particular aspect, the invention is directed to a method of inducing an immune response to HCV in an individual in need thereof comprising administering a therapeutically effective amount of a composition comprising a HCV svRNA (e.g., HCV svRNA3) that stimulates RIG-1 signaling and propagates signaling to the IFN-β gene, thereby inducing an immune response to the HCV in the individual. In particular aspects, the svRNA comprises SEQ ID NO: 1, SEQ ID NO: 3, SEQ ID NO: 4, SEQ ID NO: 25, SEQ ID NO: 26, or SEQ ID NO: 27 or combinations thereof.

In another aspect, the invention is directed to a method of treating a viral infection in an individual in need thereof, comprising administering a therapeutically effective amount of a composition comprising a (one or more) svRNA of the virus that stimulates RIG-1 signaling and propagates signaling to the IFN-β gene, thereby inducing an immune response to the virus in the individual.

In a particular aspect, the invention is directed to a method of treating a HCV in an individual in need thereof comprising administering a therapeutically effective amount of a composition comprising a HCV svRNA (e.g., HCV svRNA3) that stimulates RIG-1 signaling and propagates signaling to the IFN-β gene, thereby inducing an immune response to the HCV in the individual. In particular aspects, the svRNA comprises SEQ ID NO: 1, SEQ ID NO: 3, SEQ ID NO: 4, SEQ ID NO: 25, SEQ ID NO: 26, or SEQ ID NO: 27 or combinations thereof.

As used herein an “individual” refers to an animal, and in a particular aspect, a mammal. Examples of mammals include primates, a canine, a feline, a rodent, and the like. Specific examples include humans, dogs, cats, horses, cows, sheep, goats, rabbits, guinea pigs, rats and mice.

The term “individual in need thereof” refers to an individual who is in need of treatment or prophylaxis as determined by a researcher, veterinarian, medical doctor or other clinician. In one embodiment, an individual in need thereof is a mammal, such as a human.

The need or desire for administration according to the methods of the present invention is determined via the use of well known risk factors. The effective amount of a (one or more) particular compound is determined, in the final analysis, by the physician in charge of the case, but depends on factors such as the exact condition to be treated, the severity of the condition from which the patient suffers, the chosen route of administration, other drugs and treatments which the patient may concomitantly require, and other factors in the physician's judgment.

As used herein, “effective amount” or “therapeutically effective amount” means an amount of the active compound that will elicit the desired biological or medical response in a tissue, system, subject, or human, which includes alleviation of the symptoms, in whole or in part, of the condition being treated.

Any suitable route of administration can be used, for example, oral, dietary, topical, transdermal, rectal, parenteral (e.g., intravenous, intraarterial, intramuscular, subcutaneous injection, intradermal injection), inhalation (e.g., intrabronchial, intranasal or oral inhalation, intranasal drops), ocular, pulmonary, nasal, and the like may be employed. Administration can be local or systemic as indicated. The preferred mode of administration can vary depending on the particular agent chosen. Suitable dosage forms include tablets, troches, dispersions, suspensions, solutions, capsules, creams, ointments, aerosols, and the like.

The compound can be administered in a single dose (e.g., in a day) or in multiple doses. In addition, the compound can be administered in one or more days (e.g. over several consecutive days or non-consecutive days).

The invention is also directed to pharmaceutical compositions comprising a (one or more) nucleic acid described herein (e.g., SEQ ID NO: 1, SEQ ID NO: 3, SEQ ID NO: 4, SEQ ID NO: 25, SEQ ID NO: 26, SEQ ID NO: 27). For instance, the compositions can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other compounds.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active compound. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., that are, if desired, sterilized or mixed with auxiliary agents, e.g., preservatives, stabilizers, wetting agents, buffers or salts for influencing osmotic pressure, etc. The compound may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Compounds described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The compounds are administered in a therapeutically effective amount. The amount of compounds that will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms of an angiogenic disease, a vascular disease, a heart disease, or a circulatory disease, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, that notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the compounds can be separated, mixed together in any combination, present in a single vial or tablet. Compounds assembled in a blister pack or other dispensing means is preferred. For the purpose of this invention, unit dosage is intended to mean a dosage that is dependent on the individual pharmacodynamics of each compound and administered in FDA approved dosages in standard time courses.

### EXEMPLIFICATION

### Materials and Methods

### Mice, Cells, and Virus

Rig-i^{-/-} mice and mef and Ips1^{-/-} mef were provided by S. Akira (Osaka, Japan) (Kato et al. 2005; Kumar et al. 2006) and Mda5^{-/-} mef were provided by M. Colonna and M. Diamond (St. Louis, Mo.) (Gitlin et al. 2006). Huh7 and Huh7.5 (T55I mutant RIG-I) (Sumpter et al. 2005) human hepatoma cells, and plasmid p90/HCV FL-long pU (AF009606) encoding a full-length HCV genome, genotype 1a (strain H77), were kindly provided by C. Rice (Rockefeller University) (Blight et al. 2003). A clone of HCV JFH-1 was provided by T. Wakita (Tokyo, Japan) (Wakita et al. 2005). 293T and DU145 cells were obtained from American Type Culture Collection.

### Synthesis and Purification of HCV RNA

Subgenomic HCV RNA fragments were produced from T7 promoter-linked PCR products generated from HCV H77 plasmid. The HCV DNA was transcribed using the T7-Megascript kit according to the manufacturer's protocol (Applied Biosystems). RNA was purified according to the protocol provided with the Megaclear kit (Applied Biosystems). As an alternative to in vitro synthesis, some of the smaller RNAs (<50 nt) with either a 3′-OH or a 3′-p group were chemically synthesized at IDT, Inc. The presence of 3′-p group was confirmed by mass spectrophotometric analysis.

### Cleavage of HCV RNA by RNase L

HCV RNA (100 μg per reaction) was digested in vitro with purified recombinant human RNase L (2 μg) (Dong et al. 1994) and unfractionated 2-5A (10 μM) prepared as described (Thakur et al. 2007). Control reactions were with RNase L in the absence of 2-5A. Cleavage of the RNA was monitored in reactions that included trace amounts (0.1 nM) of an RNA FRET probe containing multiple cleavage sites for RNase L (Thakur et al. 2007). Samples were taken from 0 to 60 min at 22° C. to measure fluorescence as an indicator of RNase L activity. To generate small RNAs with 3′-p ends, the RNA were digested in vitro with purified RNase L and crude 2-5A as above. Small RNAs lacking the 3′-p or 5′-p3 were generated by incubating with 10 units CIP (NEB) for 1 h at 37° C. and 10 min for 75° C. Removal of 5′-p3 was monitored in a parallel reaction using 5′−³²P-end labeled svRNA3 and CIP. Reactions were monitored up to 1 h until removal of radiolabeled ³²P from svRNA3 was complete as monitored on a 12% sequencing gel (Malathi et al. 2007). Small RNA cleavage products (<200 nt) were purified using a solid-phase fractionation method (mirVana miRNA Isolation Kit, Ambion). To confirm complete cleavage of svRNA3, trace amounts of dephosphorylated svRNA3 and svRNA3′-p were labeled at its 5′-OH with [γ³²-P]-dATP and T4 polynucleotide kinase (NEB, MA) and separated on 12% sequencing gels and subjected to autoradiography. RNA digestions were also monitored by analysis on RNA chips (Agilent Bioanalyzer). To map the RNase L cleavage site in svRNA3, the RNA product was converted to cDNA using ExactSTART Small RNA Cloning Kit (EPICENTRE Biotechnologies) and sequenced. Briefly, the RNAs were tailed with polyA polymerase and converted to cDNA using oligo-dT₍₁₂₋₁₈₎ and MMLV reverse transcriptase (Epicentre Biotechnologies) and sequenced.

### Additional Plasmids, Reagents, and Transfection

Plasmids pEF-TAK Flag-RIG-I, pEF-TAK Flag-MDA5, recombinant full-length (RIG-I) and N-RIG (encoding amino acids 1-228) were described previously (Saito et al. 2007, 2008). In vitro transcribed HCV RNA (1 μg) or svRNAs (30 pmol or as indicated) were transfected using Lipofectamine 2000 as per maufacturer's protocol. Cells were transfected with 5 μM 2-5A complexed with Lipofectamine 2000 (Invitrogen) or poly(rI:rC) (1 μg/mL) complexed with Fugene 6 (Roche) as described previously (Malathi et al. 2007). For IFN-β promoter assays, Huh7 cells (2×105 per well in six-well plates) were transiently transfected with hIFN-β-luc (1 μg) encoding firefly luciferase cDNA under the control of the human IFN-β promoter (Sumpter et al. 2005) and a Renilla luciferase control plasmid (pRL-TK) (0.1 μg). After 24 h, 1 μg HCV RNA segment, 30 pmol of HCV svRNA3 or its derivatives, were complexed with lipofectamine 2000 and transfected. Control cells were treated with lipofectamine 2000 only. After 18 h, samples were subjected to dual luciferase assays (Promega).

### Expression and Purification of Flag-RIG-I and Flag-MDA₅

293T cells (4×10⁶) were transfected using lipofectamine 2000 (Invitrogen) with 10 μg of FLAG-tagged human RIG-I (pEF-BOS Flag-RIG-I) or human MDA5 (pEF-BOS Flag-MDA5) or vector alone. At 48 h post-transfection, cells were lysed and Flag-RIG-I or Flag-MDA5 immunoprecipitated as described (Sumpter et al. 2005; Plumet et al. 2007). To purify Flag-RIG-I or Flag-MDA5 proteins, the immunoprecipitated beads were incubated with 100 μg/mL of Flag peptide in 50 mM Tris-HCl, pH 7.4, 150 mM NaCl for 20 min at 25° C. The Flag-tagged proteins were concentrated using Microcon centrifugal devices with a 30-kDa MWCO (Millipore) and the purity of the protein determined by SDS/polyacrylamide gel electrophoresis.

Cloning and Sequencing of svRNAs

The small RNAs generated by RNase L-digestion of HCV RNA (pooled) (16 μg) were added to the immunoprecipitated Flag-RIG-I or Flag-MDA5 (1-2 μg) in 100 μL final volume. The mixtures were stirred for 4 h at 4° C., the complex was collected by a brief (30 sec) centrifugation at 2000 g. The beads were washed twice in the same buffer (500 μL) containing 100 mM NaCl. The bound RNAs were recovered after acid phenol extraction using the mirVana miRNA Isolation Kit (Ambion) and cloned using the miRCat-33 microRNA Cloning Kit Integrated DNA Technologies (IDT). The 3′ cloning linkers were ligated to small RNA species in preparation for cDNA synthesis and amplification. Reverse transcription of the linkered RNA species was followed by PCR amplification and cloning of the PCR the amplicons using TOPO-TA Cloning kit (Invitrogen). Plasmid DNA was prepared and sequenced to identify the HCV RNA fragments. Subsequently, the precise ends of the fragments were determined by comparing to the RNase L-mediated cleavage sites in HCV H77 RNA (Han et al. 2004).

### RIG-I Binding and Activation Assays by Gel-Shift Analysis and Partial Trypsin Digestion

Complex formation between 10 pmol of purified N-RIG (RIG-I amino acids 1-228, control) or full-length RIG-I (FL) and 6 pmol of indicated RNAs was determined by incubating for 15 min at 37° C. in binding buffer (20 mM Tris-HCl pH 8.0, 1.5 mM MgCl2, 1.5 mM dithiothreitol), followed by electrophoresis on a 2% agarose gel and staining with Sybr Green II RNA Gel stain kit (Lonza) (Saito et al. 2008). The gel-shift was visualized using a UV illuminator (302 nm) with a Sybr Green detection filter. Effect of RNA on RIG-I activation was determined by limited trypsin digestion of the RIG-I/RNA complex. The complex formed between 15 pmol of purified RIG-I protein and increasing amounts (3, 6, 15, or 30 pmol) of polyU/UC or svRNA3 containing the indicated ends was digested with trypsin for 15 min at 37° C. After inactivation of trypsin, one-tenth of the reaction mix was separated on 4%-15% gradient SDS polyacrylamide gel and silver-stained (Saito et al. 2008; Takahasi et al. 2008).

### ATPase Activation Assays

ATPase assays were performed in helicase buffer (25 mM Tris-HCl, pH 7.4, 3 mM dithiothreitol) in the presence of 2 mM ATP, 3 mM MgCl₂ as described (Gee et al. 2008). The standardized reactions contained 225-nM full-length Flag-RIG-I in a 20 reaction at 37° C. typically ranging from 5 to 90 min. Reaction samples were stopped by rapid dilution (20-fold) in acidic malachite green solution (Cytoskeleton) supplemented with 10 mM EDTA and incubated for 15 min, and the absorbance was determined at 650 nM.

### Western Blots

Expression of Flag-hRNase L (48 h post-transfection) and HCV core protein (48 and 96 h post-electroporation) was determined on immunoblots using anti-FLAG monoclonal antibody (Sigma-Aldrich) or mouse monoclonal anti-HCV core antibody (Affinity Bioreagent, C7-50). Levels of expression of RNase L in Huh7, Huh7.5, and DU145 cells were determined on immunoblots using 30 μg of total cell lysates probed with anti-hRNase L monoclonal antibody (Dong and Silverman 1995). All secondary antibodies were purchased from Cell Signaling. Immunoreactive bands were detected using ECL reagents (GE Healthcare).

### Hydrodynamic Tail Vein Injections of Mice

Two hundred μg of poly-U/UC (5′-p3/3′-OH) or svRNA3 (5′-OH/3′-p) RNA in PBS (50 μL) were mixed with 40 μL of transfection reagent (Altogen) and incubated 15-20 min at room temperature. A transfection enhancer reagent (10 μL) was added, vortexed gently, and incubated 10 min at room temperature. Two mL of 5% glucose was added and the solution was injected into the tail vein (Saito et al. 2008).

### Immunohistochemistry

Mouse livers fixed in 4% paraformaldehyde were sectioned and immunostained using 1:1000 dilution of anti-mouse ISG54 antibody (provided by Dr. G. Sen, Cleveland Clinic) by the histology core at Cleveland Clinic. The sections were counterstained with hematoxylin. Liver biopsies recovered from patients with chronic hepatitis C virus infection (viral genotype 1b) were processed for immunostaining using monoclonal antibody specific to RNase L (Dong and Silverman 1995), OAS1 monoclonal antibody (a kind gift from Dr. Shawn Iadonato, Kineta, Inc. Seattle, Wash.), and polyclonal anti-NS5A (a gift from Dr. Jin Ye, University of Texas Southwestern Medical Center).

### Quantitative RT-PCR Analysis

Mouse liver RNA was extracted from tissue soaked in RNAlater reagent (Ambion) using RNeasy kit (Qiagen). One-step quantitative RT-PCR was performed using Applied Biosystems TaqMan Universal PCR master mix containing gene-specific primers for mouse Ifnb, rig-i, and isg56 and TaqMan probe (sequences shown in Supplemental Table S1). PCR was performed with an Applied Biosystems 7500 instrument and all data were presented as relative expression units after normalization to Gapdh mRNA.

### ELISAs

Murine IFN-β from WT, Rig-i^{-/-} mice, and culture supernatants derived from MEFs were measured by using ELISA kits purchased from PBL Biomedical Laboratories.

Detection of svRNA₃ in Intact Cells

Huh7.5 cells (1×10⁷) were electroporated with 30 μg of in vitro transcribed full-length HCV 1a RNA, RNA corresponding to nt 8703-9416 or mock treated using 0.4-cm gap cuvette (0.22 kV, 960 μF) and Gene Pulsar II from Bio-Rad. After 24 h, 8 μg of plasmid Flag-hRNase L was transfected using Fugene 6 reagent as per manufacturer's protocol. After 48 h (72 h post-electroporation), cells were treated with IFN-β (1000 IU/mL) and incubated for another 18 h. Poly(rI:rC) (1 μg/mL) or 5 μM of 2-5A was transfected using Fugene6 or lipofectamine 2000 reagent, respectively, for 6 h. Cell lysates were prepared from aliquots of samples for immunodetection of Flag-hRNase L and HCV core protein. Total RNA was isolated using the TRIZOL reagent. To monitor RNase L cleavage of RNA, RNA (4 μg) was separated and analyzed on RNA chips (Agilent BioAnalyzer) to monitor activation of RNase L. Small RNAs (<200 nt) were purified using a solid-phase fractionation method (mirVana miRNA Isolation Kit, Ambion). Small RNA (150 μg) from different treatments and 1 ng of svRNA3 (5′-OH/3′-p) was electrophoresed on 8% PAGE. The RNAs were transferred to BrightStar-Plus membrane (Ambion) and immobilized by UV cross-linking. Probe was synthesized using miRNA StarFire System corresponding to the sequence of svRNA3 (5′-gaaccaaccggacaagtccagccggccagcggccgctattggag-3′) with [α-32P]-dATP (6000 Ci/mmol, Perkin Elmer). Hybridization was done at 42° C. in ULTRAhyb-Oligo Hybridization buffer (Ambion) for 18 h. RNA samples (10 μg) and 100 ng of svRNA3 (5′-OH/3′-p) were stained with Gel Star Nucleic acid stain (Lonza) to compare loading of the samples.

### Results

Identification of HCV RNA Cleavage Products that Bind RIG-I

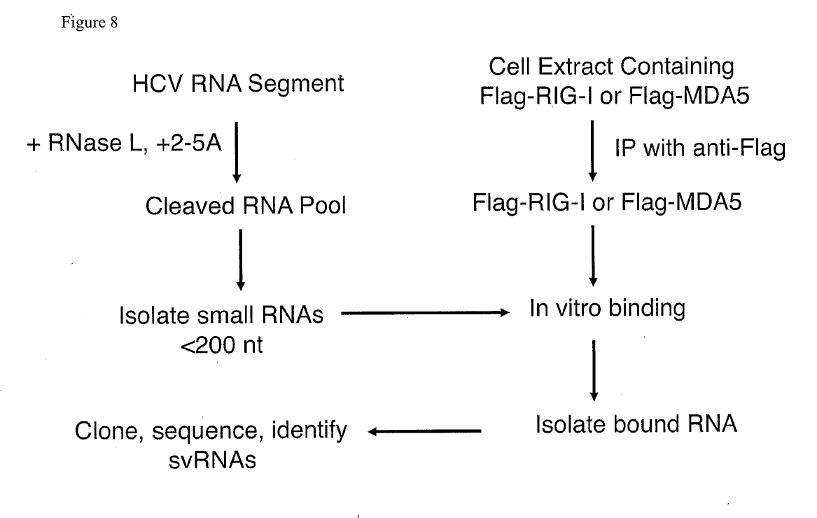

Whether RNase L processes HCV genomic RNA into small RNAs with PAMP activity (designated “suppressor of virus RNA” or svRNA) was investigated. HCV RNA was selected as a substrate because the RNase L cleavage sites had been previously determined (Han et al. 2004), thus allowing the termini of cleavage products to be precisely mapped. In addition, an M-fold secondary structure prediction of the entire HCV H77 genomic RNA (performed as in Palmenberg and Sgro 1997) was used to identify structural domains in the HCV genomic RNA. Based on both known and predicted structural features in the HCV RNA, eight regions spanning the entire genome as substrates for digestion by RNase L were selected (FIG. 1A, lower). The eight HCV RNA segments were generated by in vitro transcription, purified, and cleaved by RNase L. RNA cleavage products <200 nucleotides (nt) in length were isolated, bound to flag tagged-RIG-I or -MDA5, and cloned (FIG. 8). Fifteen small RNAs with affinity for RIG-I were cloned, while no clones were obtained using MDA5 (Table 1). It was apparent from the comparison of the cloned sequences with previously determined RNase L cleavage sites (Han et al. 2004) that all were partial clones of the small RNAs (Table 2).

Prior to investigating the small RNAs, whether the uncleaved HCV RNA segments had PAMP activity was determined. The RNAs were individually transfected into human hepatoma Huh7 cells containing the human IFN-β promoter fused to firefly luciferase cDNA. As reported previously (Saito et al. 2008), some of these RNA fragments induced the IFN-β promoter, especially fragments 8703-9416 and 8703-9646 nt, which contains the poly-U/UC region (9406-9547 nt) (FIG. 1A). In contrast, the small RNAs generated using RNase L had little or no ability to induce the IFN-β promoter, except for the small RNAs generated from fragments 8703-9416 and 8703-9646 nt (FIG. 1A). Remarkably, cleavage of the 8703-9416 nt fragment with RNase L caused a large increase in the ability to induce the IFN-β promoter, whereas RNase L cleavage of the 3′-extended fragment (8703-9646 nt) containing the poly-U/UC region resulted in a modest increase in PAMP activity (p<0.0006 and p<0.038, respectively). In contrast, PAMP activity associated with poly-U/UC was destroyed by digestion with RNase L because of the sequence preferences of RNase L for UU and UA dinucleotides in single-stranded RNA (FIG. 1A, rightmost pair of columns). The RNA fragment (8703-9416 nt) that produced the highest level of PAMP activity upon RNase L cleavage yielded three clones of RIG-I-bound RNAs: svRNA3, svRNA14, and svRNA15 (Tables 1, 2). SvRNA14 and svRNA15 originated from an identical 82-nt fragment of the NS5B gene, but lacked significant structure with a maximum of four predicted consecutive base pairs (as determined using MFOLD) (Zuker 2003), and were not pursued further. In contrast, svRNA3 (9192-9281 nt also from the NS5B gene) is a highly structured RNA, including a stretch of 11 consecutive base pairs. Cleavage of HCV RNA by RNase L at nt 9191 (UA9191) and nt 9281 (UU9281) releases a 90-base fragment of HCV RNA from the NS5B coding region that refolds into svRNA3 (FIG. 1B).

SvRNA3 is Formed from HCV Genomic RNA Through the Action of RNase L in Intact Human Hepatoma Cells

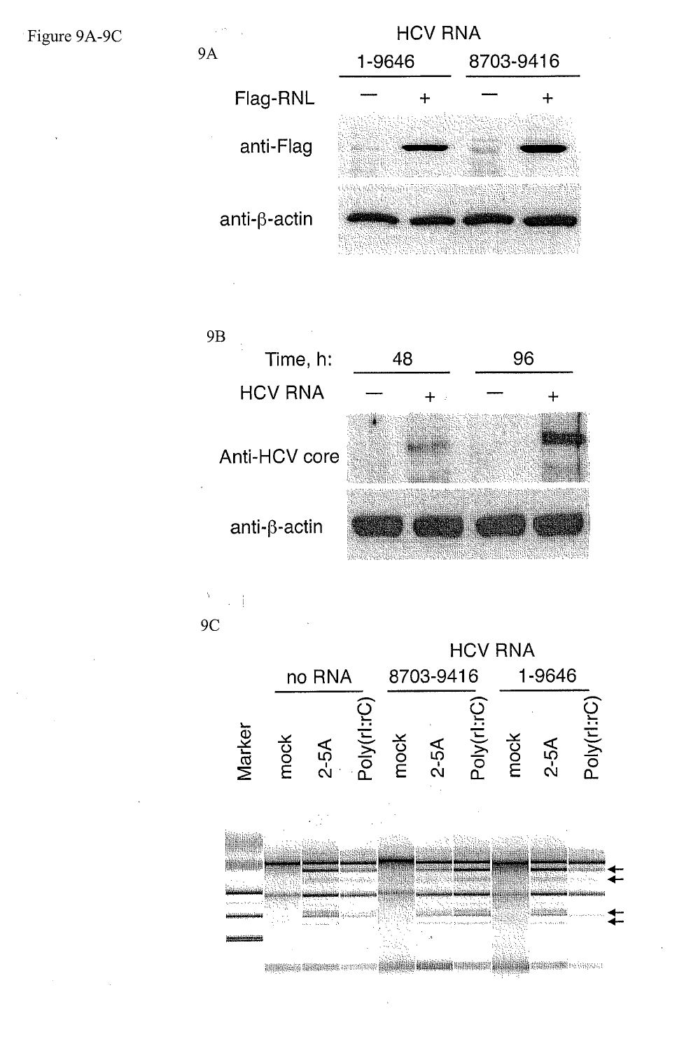

To establish if svRNA3 could be demonstrated to form in intact cells, full-length genomic RNA or HCV RNA fragment 8703-9416 nt, both from HCV 1a, strain H77, were electroporated into Huh7.5 cells. After 24 h, transfection of flag-RNase L cDNA was performed to elevate levels of RNase L followed by treatment with IFN-β to elevate OAS levels (FIG. 9A). Production of HCV core antigen was observed in cells containing full-length HCV RNA demonstrating translation and processing of the HCV polyprotein, (FIG. 9B). Furthermore, RNase L activation was observed in flag-RNase L expressing cells as measured by the appearance in RNA chips of specific and characteristic rRNA cleavage products when cells were treated with either 2-5A (activator of RNase L) or poly(rI:rC) (activator of OAS) (FIG. 9C; Silverman et al. 1983). It was observed that svRNA3 was generated from HCV RNA in the intact cells upon activation of RNase L by 2-5A or poly(rI):(rC) as determined in Northern blots (FIG. 1C). Results showed that HCV RNA (either the full-length genomic RNA or fragment 8703-9416) is processed into svRNA3 when OAS and RNase L are present and active.

Activation of the RNA Helicase, RIG-I, by svRNA3 is Dependent on its 3′-p Group

A method was devised for producing svRNA3 (5′-OH/3′-p) in which a precursor (with 5′-p₃ and a 3′-extension of UUA) was synthesized with T7 RNA polymerase (FIG. 2A). The 5′-p₃ was removed with calf intestinal phosphatase (CIP), while the UUA sequence, and any possible aberrant extension of the 3′-terminus (Cazenave and Uhlenbeck 1994; Schlee et al. 2009), was removed by RNase L, which also generated a 3′-p (FIG. 2B). In every instance, 5′-p₃/3′-OH svRNA3 refers to the precursor, whereas 5′-OH/3′-p is the mature RNase L product. In addition, to determine the minimal structural requirements in svRNA3 that contribute to signaling, we also generated derivatives of svRNA3 (5′-p₃ forms): a complementary form (CsvRNA3), an isolated stem-loop (svRNA3 short), and deletion of the 5′, 3′, or both overhangs (5′-A svRNA3, 3′-A svRNA3, and 5′-, 3′-A svRNA3)) (FIG. 2C-G). Activation of the ATPase function of RIG-I was obtained with poly-U/UC (5′-p₃/3′-OH) and with svRNA3 (5′-p₃/3′-OH or 5′-OH/3′-p), but not with the 5′-OH/3′-OH forms of these RNAs (FIG. 2H). Interestingly, the 5′-OH/3′-p and 5′-p₃/3′-OH forms of svRNA3 were equally active in this assay. Weak activation of RIG-I ATPase was also obtained with the 5′- and 3′-deleted forms of svRNA3 (5′-p₃/3′-OH), but not with any of the other RNAs. The only RNA that activated the MDA5 ATPase, albeit modestly, was poly(rI):(rC) (FIG. 2H).

The 5′-OH/3′-p form of svRNA3 stimulated the IFN-β promoter activity in Huh7 cells to 190% of the level obtained with the 5′-p₃/3′-OH form, whereas the 5′-OH/3′-OH form was inactive (FIG. 21). The 5′-p₃/3′-OH form of poly-U/UC stimulated the IFN-β promoter to 155% of the level obtained with 5′-OH/3′-p svRNA3. These findings show that a 3′-p group on svRNA3 could effectively substitute for a 5′-p3 group to signal to the IFN-β gene. None of the other RNAs induced IFN-β promoter activity. These results indicate that 5′- and 3′-ss regions of svRNA3 and both the upper and lower stems were required for optimal RIG-I activation.

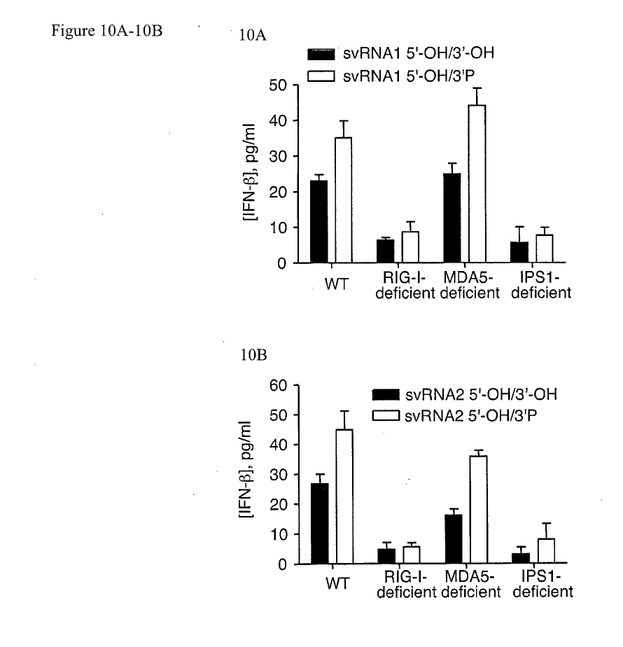

IFN-β induction was compared in wild-type (WT) and gene deficient mouse embryo fibroblasts (mef) treated with svRNAs1, -2 (FIGS. 10A-10B), and svRNA3 (FIG. 2J) (all with 5′-OH/3′-p termini). While svRNA1, svRNA2, and svRNA3 all required RIG-I and IPS-1 expression (but not MDA5) for IFN-β induction, svRNA3 was about 30-fold more active. Furthermore, the 5′-p₃/3′-OH and 5′-OH/3′-p versions of svRNA3 were equally potent PAMPs in WT mef (FIG. 2J).

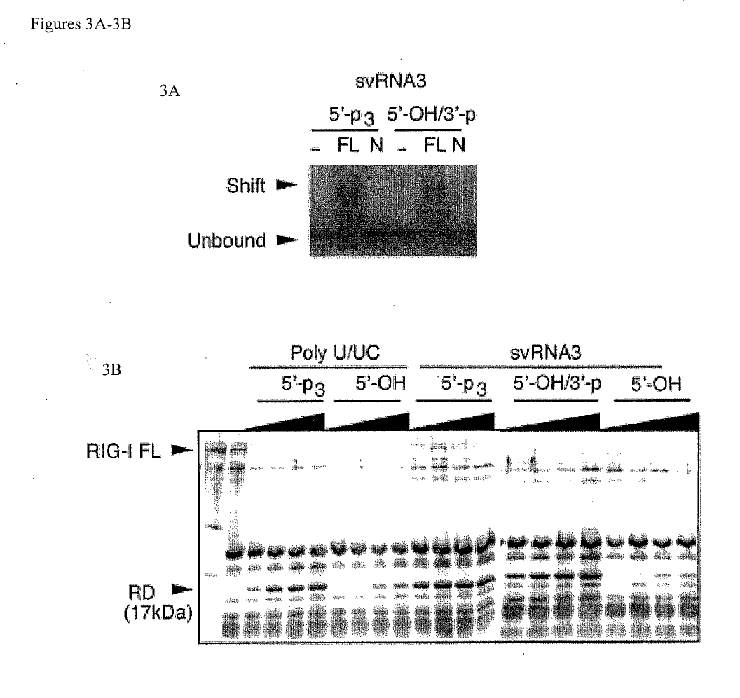

Viral RNA PAMPs that signal through RIG-I induce conformational changes that displace the C-terminal repressor domain (RD) allowing interaction with the adapter protein, IPS-1 (Saito et al. 2007). SvRNA3 with either 5′-p₃/3′-OH or with 5′-OH/3′-p formed a stable complex with full-length RIG-I, but not with an N-terminal polypeptide of RIG-I (FIG. 3A). Efficient displacement of the RIG-I RD was obtained with 5′-p₃-polyU/UC and either 5′-p₃ or 3′-p forms of svRNA3 (FIG. 3B). The dephosphorylated forms of these RNAs lacked activity.

### SvRNA3 Induces a Hepatic Innate Immune Response in Mice

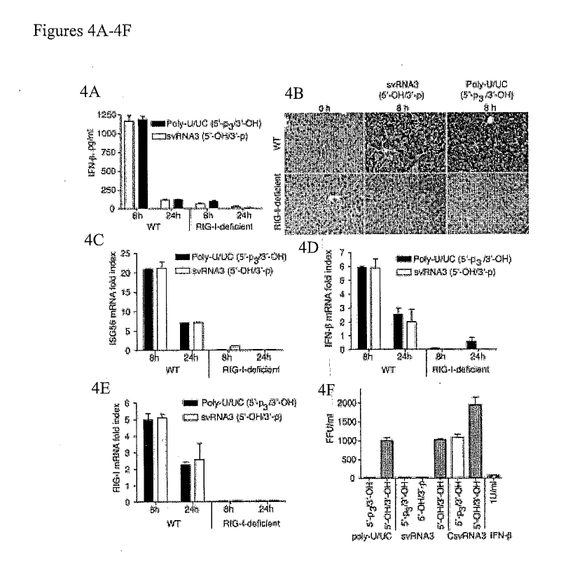

To study the effects of svRNA3 on hepatic innate immunity in vivo, WT or RIG-I-deficient mice were treated by hydrodynamic tail vein injection with either svRNA3 (5′-OH/3′-p) or, as a positive control, poly-U/UC (5′-p₃/3′-OH). This procedure efficiently introduces the HCV RNA and its subgenomic counterparts into the mouse hepatocytes, thus modeling the viral RNA-host interactions of an acute HCV infection (Saito et al. 2008). Both svRNA3 (5′-OH/3′-p) and the poly-U/UC (5′-p₃/3′-OH) RNA were equally potent in the RIG-I-dependent induction of IFN-β by 8 h in WT, but not in RIG-I-deficient mice, as determined by specific ELISAs performed on sera (FIG. 4A). By 24 h post-treatment circulating IFN-β levels returned to basal levels. A dramatic induction of ISG54 protein was observed in the liver after 8 h of treatment with svRNA3 or poly-U/UC in WT mice, but not in RIG-I-deficient mice (FIG. 4B) indicative of paracrine signaling of IFN-β. In addition, levels of mRNAs for ISG56, IFN-β, and RIG-I were highly induced at 8 h in WT, but not in RIG-I-deficient mice, and were declining by 24 h post-treatment with either svRNA3 or poly-U/UC (FIG. 4C-E).

### SvRNA3 or 2-5A Inhibits HCV Replication in Huh₇ Cells

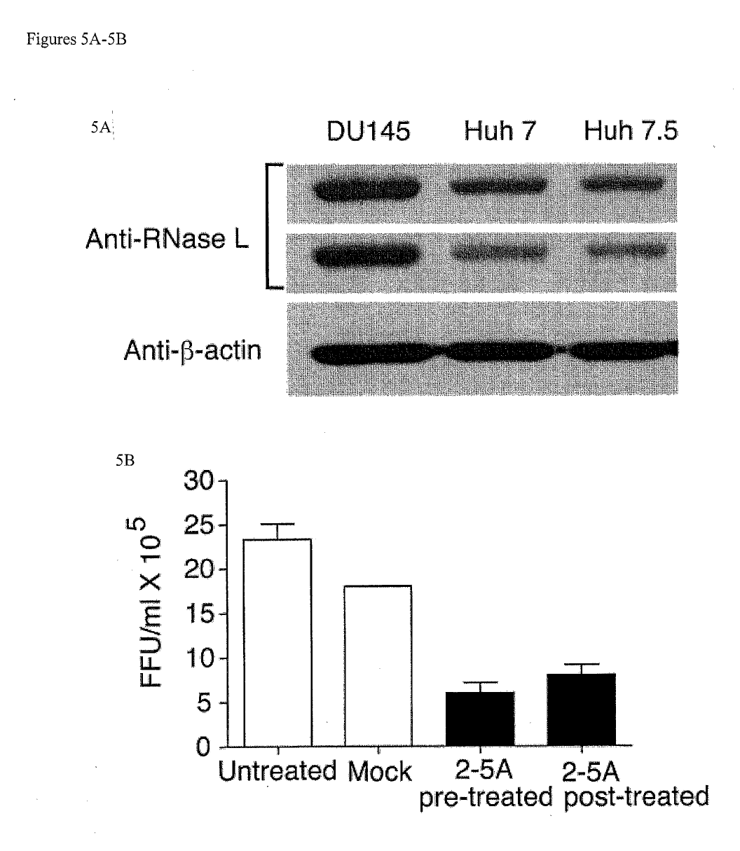

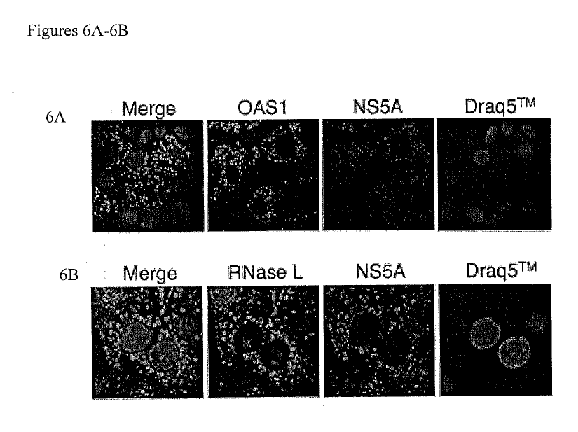

To determine if svRNA3 could inhibit HCV replication in vitro through a paracrine mechanism, conditioned media from Huh7 cells transfected with svRNA3, CsvRNA3, or poly-U/UC were added to Huh7.5 cells (harboring a defective form of RIG-I) (Sumpter et al. 2005) for 12 h prior to infection with HCV strain JFH-1 for 48 h (Wakita et al. 2005). Focus-forming unit (FFU) assays with anti-HCV antibody showed antiviral activity with poly-U/UC (5′-p₃/3′-OH) and svRNA3 (5′-p₃/3′-OH or 5′-OH/3′-p), but not with the same RNAs containing 5′-OH and 3′-OH termini (FIG. 4F). The complement of svRNA3, CsvRNA3, was inactive in these assays, while IFN-β, included as a positive control, inhibited HCV infection. In addition, direct activation of RNase L in Huh7 cells reduced HCV yields to 33%±11 (p=0.0091) of that obtained for the control infected cells as measured by FFU, even though these cells have a relatively low level of RNase L (FIGS. 5A-5B). Presumably, the anti-HCV effect would be even greater in human hepatocytes in vivo, which are known to express RNase L at higher levels (Zhou et al. 2005). Therefore OAS1 and RNase L expression in human HCV patients were evaluated via confocal microscopy analysis of immunostained liver sections. Both OAS1 and RNase L were abundant in HCV-infected hepatocytes from patients (FIGS. 6A-6B). The merged images indicate, but do not prove, colocalization with the viral NS5A protein. However, NS5A does mark intracellular sites of HCV replication and was previously shown to be a binding partner of OAS1 (Taguchi et al. 2004; Wakita et al. 2005).

### Discussion

The results provided herein define features in an RNase L-mediated RNA cleavage product necessary for RIG-I activation. The finding of a previously unrecognized role for a 3′-p in RIG-I activation, effectively substituting for a 5′-p₃ group, is a novel paradigm for an RNA PAMP. The predicted structure of svRNA3 includes a broken-stem-loop with 5′ and 3′overhangs, both of which contribute to the activation of RIG-I. Surprisingly, the main double-stranded stem of svRNA3 (svRNA3 short) including a 3′-p lacked PAMP activity. Results indicate that in addition to 3′-p a higher order structure is necessary for recognition by and activation of RIG-I. For instance, it was observed that svRNA4 (65 nt) bearing 3′-p or 5′-p₃ did not stimulate RIG-I activity.

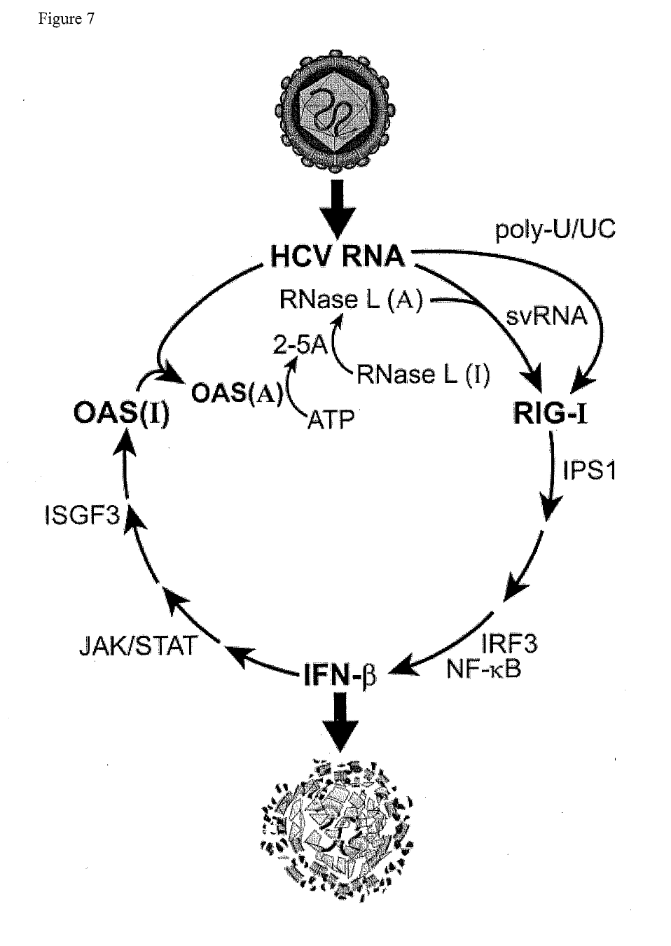

These findings indicate the following scenario for innate immunity against HCV (FIG. 7). At early times of infection, RIG-I detects and responds to the HCV PAMP, poly-U/UC in the HCV 3′-UTR in concert with a 5′-p₃ group, leading to activation of IRF3 and synthesis of type I IFN (Saito et al. 2008). IFN-β induces tissue-wide transcriptional activation of OAS genes and viral dsRNA activates OAS to produce 2-5A (Han and Barton 2002), which activates RNase L. Specifically, RNase L cleaves single stranded (ss) regions of HCV RNA on the 3′ side of UUp and UAp dinucleotides, including destroying the poly-U/UC PAMP (FIG. 1A). While it may be premature to conclude that svRNA will amplify innate immune signaling against HCV, the data do indicate this. However, in addition, the intracellular host innate immune response is blunted by the action of different HCV proteins, including the NS3/4A protease complex that cleaves IPS-1, the HCV core protein that binds STAT1, and NS5A protein that suppresses activation of PKR (for review, see Horner and Gale 2009).

RNase L cleaves at sites throughout the HCV genomic RNA producing many small RNAs, but svRNA3 is by far the most active PAMP that is released during this process. Remarkably, IFN sensitivity of HCV strains in vivo nevertheless correlates with the number of potential RNase L cleavage sites (Han et al. 2004). Accordingly, there are fewer potential RNase L cleavage sites in IFN-resistant genotype 1a and 1b compared to IFN-sensitive genotypes 2a, 2b, 3a, and 3b (Han and Barton 2002). The findings provided herein indicate that an RNase L cleavage product of HCV RNA is able to stimulate RIG-I signaling. SvRNA3, composed of RNA sequences from two adjacent stem-loop structures within the NS5B portion of the HCV open reading frame, refolds into a RIG-I activator after its release by RNase L (FIG. 1B). HCV1a RNA is cleaved efficiently by RNase L at UA₉₁₉₁, UU₉₂₈₁ UU₉₂₈₂, and UA₉₂₈₃ (Han et al. 2004), liberating svRNA3 with a 3′ phosphoryl group (FIG. 1B). Importantly, RNA sequences and structures in this region of HCV RNA are phylogenetically conserved and they are required for HCV RNA replication (Tuplin et al. 2002, 2004; Lee et al. 2004; You et al. 2004; Diviney et al. 2008). Single-stranded UA and UU dinucleotides are phylogenetically conserved at nt 9281-9283 in HCV genotypes 1, 2, 3, 4, 5, and 6 (Diviney et al. 2008, FIG. 6A). The ₉₂₈₄UCACAGC₉₂₉₀ sequences immediately adjacent to these single-stranded UA and UU dinucleotides are invariant in HCV genotypes 1-6 and they form a kissing interaction with complementary sequences in the HCV 3′ NTR (Diviney et al. 2008, FIG. 8). The ₉₃₀₀GCCCG₉₃₀₄ sequences in the subsequent loop are also invariant in HCV genotypes 1-6 and are predicted to form a pseudoknot via base-pairing to ₉₁₀₈CGGGC₉₁₁₂ sequences upstream in the NS5B ORF (Diviney et al. 2008). The RNA sequences and structures in this region of HCV NS5B are well conserved across all six HCV genotypes.

Finally, because RNase L has a relatively broad antiviral activity for many RNA viruses (Silverman 2007), chemical features of svRNA such as, but not limited to, the 3′-p group, or methods that will activate RNase L in vivo may lead to broad-spectrum antiviral agents active against HCV and other important viral pathogens.

TABLE 1

| | | | | | SEQ. |
|---|---|---|---|---|---|
| | | | | Clones | ID |
| Name | HCV Seqeuence | HCV nt | Genomic Region | Isolated | NO. |
| svRNA1 | acctccaccaga | 2413-2424 | p7(ion channel)/NS2 | six | 10 |
| | | | (cysteine protease) | | |
| svRNA2 | acgctgggctttgg | 4122-4135 | NS3 (serine protease/RNA helicase) | three | 11 |
| svRNA3 | aaactcactccaatagcg | 9204-9221 | NS5B | eight | 12 |
| svRNA4 | gggtgtgcgcgcgacgaggaagacttccga | 473-536 | C (core) | one | 13 |
| | gcggtcgcaacctcgaggtagacgtcagcc | | | | |
| | tatc | | | | |
| svRNA5 | ggcgccactggacg | 1228-1241 | E1 glycoprotein | one | 14 |
| svRNA6 | tgatcgctggtgctcactggggag | 1381-1404 | E1 glycoprotein | two | 15 |
| svRNA7 | ccggctggttag | 1645-1656 | E2 glycoprotein | two | 16 |
| svRNA8 | aggggtggaggttg | 3403-3416 | N52/NS3 (serine protease/RNA helicase) | one | 17 |
| syRNA9 | acggcgtacgc | 3429-3439 | N52/N53 (serine protease/RNA helicase) | one | 18 |
| svRNA10 | acacgccgtgggcctat | 3863-3879 | NS2/NS3 (serine protease/RNA helicase) | one | 19 |
| svRNA11 | aacacctgggtgc | 5313-5324 | NS4A (serine protease cofactor)/NS4B | one | 20 |
| | | | (membrane alterations) | | |
| svRNA12 | caggagatgggcg | 7044-7056 | NS5A (phosphoprotein) | two | 21 |
| svRNA13 | cctgtgcctccgcctcgg | 7308-7325 | NS5A (phosphoprotein)/N 513 (RdRP) | one | 22 |
| svRNA14 | accctacaaccccc | 8761-8775 | NS58 (RdRP) | one | 23 |
| svRNA15 | ctggctaggcaac | 8822-8834 | NS58 (RdRP) | two | 24 |

TABLE 2

| | | | SEQ |
|---|---|---|---|
| | | | ID |
| Name | HCV nt | Sequences | NO. |
| svRNA1 | 2395-2429 | UGUCCACCGGCCUCAUCCACCUCCACCAGAAUU | 25 |
| svRNA2 | 4112-4140 | UGUUGCUGCAACGCUGGGCUUUGGUGCUU | 26 |
| svRNA3 | 9191-9281 | AAGAACAAAGCUCAAACUCACUCCAAUAGCGGCCGCUGGCCGGCUGGACUUGUCCGGUUGGUUC | 27 |
| | | ACGGCUGGCUACAGCGGGGGAGACAU | |
| svRNA4 | 472-536 | UGGGUGUGCGCGCGACGAGGAAGACUUCCGAGCGGUCGCAACCUCGAGGUAGACGUCAGCCUAUC | 28 |
| svRNA5 | 1215-1257 | ACCUUCUCUCCCAGGCGCCACUGGACGACGCAAGACUGCAAU | 29 |
| svRNA6 | 1280-1423 | ACGGGUCAUCGCAUGGCAUGGGAUAUGAUGAUGAACUGGUCCCCUACGGCAGCGUUGGUGGUAG | 30 |
| | | CUCAGCUGCUCCGGAUCCCACAAGCCAUCAUGGACAUGAUCGCUGGUGCUCACUGGGGAGUCC | |
| | | UGGCGGGCAUAGCGU | |
| svRNA7 | 1542-1755 | GGUCUCCUUACACCAGGCGCCAAGCAGAACAUCCAACUGAUCAACACCAACGGCAGUUGGCACAUCA | |
| | | AUAGCACGGC | |
| | | CUUGAAUUGCAAUGAAAGCCUUAACACCGGCUGGUUAGCAGGGCUCUUCUAUCAACACAAAUUCAA | 31 |
| | | CUCUUCAGGCUGUCCUGAGAGGUUGGCCAGCUGCCGACGCCUUACCGAUUUUGCCCAGGGCUG | |
| | | GGGUCCUA | |
| svRNA8 | 3374-3470 | GCUUGGGCCAGCCGACGGAAUGGUCUCCAAGGGGUGGAGGUUGCUGGCGCCCAUCACGGCGUA | 32 |
| | | CGCCCAGCAGACGAGAGGCCUCCUAGGGUGUAUA | |
| svRNA9 | 3374-3470 | GCUUGGGCCAGCCGACGGAAUGGUCUCCAAGGGGUGGAGGUUGCUGGCGCCCAUCACGGCGU | 33 |
| | | ACGCCCAGCAGACGAGAGGCCUCCUAGGGUGUAUA | |
| svRNA10 | 3733-3912 | ACCUGGUCACGAGGCACGCCGAUGUCAUUCCCGUGCGCCGGCGAGGUGAUAGCAGGGGUAGCC | 34 |
| | | UGCUUUCGCCCCGGCCCAUUUCCUACUUGAAAGGCUCCUCGGGGGGUCCGCUGUUGUGCC | |
| | | CCGCGCGACACGCCGUGGGCCUAUUCAGGGCCGCGGUGUGCACCCGUGGAGUGGCU | |
| svRNA11 | 5218-5399 | ACAGACUGGGCGCUGUUCAGAAUGAAGUCACCCUGACGCACCCAAUCACCAAAUACAUCAUGACA | 35 |
| | | UGCAUGUCGGCCGACCUGGAGAUCGUCACGAGCACCUGGGUGCUCGUUGGCGGCGUCCUGG | |
| | | CUGCUCUGGCCGCGUAUUGCCUGUCAACAGGCUGCGUGGUCAUAGUGGGCAGGATT | |
| svRNA12 | 6984-7232 | UGCACCGCCAACCAUGACUCCCCUGACGCCGAGCUCAUAGAGGCUAACCUCCUGUGGAGGCAGG | 36 |
| | | AGAUGGGCGGCAACAUCACCAGGGUUGAGUCAGAGAACAAAGUGGUGAUUCUGGACUCCUUC | |
| | | GAUCCGCUUGUGGCAGACGAGGAUGAGCGGGAGGUCUCCGUACCUGCAGAAAUUCUGCGGA | |
| | | AGUCUCGGAGAUUCGCCCGGGCCCUGCCCGUCUGGGCGCGGCCGGACUACAACCCCCCGCUA | |
| svRNA13 | 7289-7361 | CCACCUCCACGGUCCCCUCCUGUGCCUCCGCCUCGGAAAAAGCGUACGGUGGUCCUCACCGAA | 37 |
| | | UCAACCCUA | |
| svRNA14 | 8754-8837 | CCCGUGACCCUACAACCCCCCUCGCGAGAGCCGCGUGGGAGACAGCAAGACACACUCCAGUCAAU | 38 |
| | | UCCUGGCUAGGCAACAU | |
| svRNA15 | 8754-8837 | CCCGUGACCCUACAACCCCCCUCGCGAGAGCCGCGUGGGAGACAGCAAGACACACUCCAGUCAA | 39 |
| | | UUCCUGGCUAGGCAACAU | |

TABLE 3
Oligonucleotide primers*.

| | | SEQ. |
|---|---|---|
| Oligonucleotide | 5′ - 3′ Sequence | ID NO. |
| HCV1up | GTA ATA CGA CTC ACT ATA GGG gcc agc ccc ctg atg ggg gcg aca | 40 |
| HCV2318dn | cct gtc cct gtc ttc cag atc aca gcg | 41 |
| HCV2316up | GTA ATA CGA CTC ACT ATA GGG agg tcc gag ctc agc cca ttg ctg | 42 |
| HCV3948dn | cac tag gtt ctc cac agg gat aaa gtc | 43 |
| HCV3946up | GTA ATA CGA CTC ACT ATA GGG gag aca acc atg aga tcc ccg gtg | 44 |
| HCV5435dn | cct gtc agg tat aat tgc cgg ctt ccc | 45 |
| HCV5430up | GTA ATA CGA CTC ACT ATA GGG gac agg gag gtt ctc tac cag gag | 46 |
| HCV6698dn | ggg cga tgg gat ctg gca cgg gca | 47 |
| HCV6696up | GTA ATA CGA CTC ACT ATA GGG ccc gaa ttt ttc aca gaa ttg gac | 48 |
| HCV8702dn | gga gca tga tgt tat aag ctc caa | 49 |
| HCV8703up | GTA ATA CGA CTC ACT ATA GGG tcc aac gtg tca gtc gcc cac gac | 50 |
| HCV9702dn | cgc ggc ata ccc cgc gta ttc cca | 51 |
| HCV9416dn | gga aat ggc cta aga ggc cgg agt | 52 |
| svRNA3up | GTA ATA CGA CTC ACT ATA GGG aag aac aaa gct caa act cac tc | 53 |
| svRNA3dn | taa atg tct ccc ccg ctg tag cc | 54 |
| csvRNA3up | GTA ATA CGA CTC ACT ATA GGG taa atg tct ccc ccg ctg tag cc | 55 |
| csvRNA3dn | aag aac aaa gct caa act cac tcc | 56 |
| polyU/UCup | GTA ATA CGA CTC ACT ATA GGG agg cca ttt cct gtt ttt ttt ttt | 57 |
| polyU/UCdn | aaa gaa gga agg aaa aga aag gaa a | 58 |
| svRNA3 5ssdel up | GTA ATA CGA CTC ACT ATA GGG ctc act cca ata gcg gcc gct gg | 59 |
| svRNA3 3ssdel dn | ctc ccc cgc tgt agc cag ccg tga a | 60 |
| svRNA4up | GTA ATA CGA CTC ACT ATA GGG ggg tgt gcg cgc gac gag gaa ga | 61 |
| svRNA4dn | gat agg ctg acg tct acc tcg agg | 62 |
| mIFN-b probe | \56-FAM\ATGGAAAGATCAACCTCACCTACAGGGCG\3BHQ_1\ | 63 |
| mRIG-l probe | \56-FAM\CACAAACTTGGAGAGTCACGGGACCC\3BHQ_1\ | 64 |
| mISG56 probe | \56-FAM\TCCCAACTGAGGACATCCCGAAACA\3BHQ_1\ | 65 |
| mIFN-b fwd | GAAAGGACGAACATTCGGAAAT | 66 |
| mIFN-b rev | TCCGTCATCTCCATAGGGATCT | 67 |
| mRIG-l fwd | GCAAAAACCACCCATACAATCA | 68 |
| mRIG-l rev | CGCGGTCTTAGCATCTCCAA | 69 |
| mISG56 fwd | AGGGCTCTGCTACAAGCAACA | 70 |
| mISG56 rev | TGCCAATTCTTGCACATTGTC | 71 |
| Ext up | aag aac aaa gct caa ctc act | 72 |

### REFERENCES

Armstrong G L, Wasley A, Simard E P, McQuillan G M, Kuhnert W L, Alter M J. 2006. The prevalence of hepatitis C virus infection in the United States, 1999 through 2002. Ann Intern Med 144: 705-714.
Blight K J, McKeating J A, Marcotrigiano J, Rice C M. 2003. Efficient replication of hepatitis C virus genotype 1a RNAs in cell culture. J Virol 77: 3181-3190.
Cazenave C, Uhlenbeck O C. 1994. RNA template-directed RNA synthesis by T7 RNA polymerase. Proc Natl Acad Sci 91: 6972-6976.
Cole J L, Carroll S S, Kuo L C. 1996. Stoichiometry of 2′,5′-oligoadenylate-induced dimerization of ribonuclease L. A sedimentation equilibrium study. J Biol Chem 271: 3979-3981.
Diviney S, Tuplin A, Struthers M, Armstrong V, Elliott R M, Simmonds P, Evans D J. 2008. A hepatitis C virus cis-acting replication element forms a long-range RNA-RNA interaction with upstream RNA sequences in NS5B. J Virol 82: 9008-9022.
Dong B, Silverman R H. 1995.2-5A-dependent RNase molecules dimerize during activation by 2-5A. J Biol Chem 270: 4133-4137.
Dong B, Xu L, Zhou A, Hassel B A, Lee X, Torrence P F, Silverman R H. 1994. Intrinsic molecular activities of the interferon-induced 2-5A-dependent RNase. J Biol Chem 269: 14153-14158.
Floyd-Smith G, Slattery E, Lengyel P. 1981. Interferon action: RNA cleavage pattern of a (2′-5′)oligoadenylate-dependent endonuclease. Science 212: 1030-1032.
Gee P, Chua P K, Gevorkyan J, Klumpp K, Najera I, Swinney D C, Deval J. 2008. Essential role of the N-terminal domain in the regulation of RIG-I ATPase activity. J Biol Chem 283: 9488-9496.
Gitlin L, Barchet W, Gilfillan S, Cella M, Beutler B, Flavell R A, Diamond M S, Colonna M. 2006. Essential role of mda-5 in type I IFN responses to polyriboinosinic:polyribocytidylic acid and encephalomyocarditis picornavirus. Proc Natl Acad Sci 103: 8459-8464.
Han J Q, Barton D J. 2002. Activation and evasion of the antiviral 2′-5′ oligoadenylate synthetase/ribonuclease L pathway by hepatitis C virus mRNA. RNA 8: 512-525.
Han J Q, Wroblewski G, Xu Z, Silverman R H, Barton D J. 2004. Sensitivity of hepatitis C virus RNA to the antiviral enzyme ribonuclease L is determined by a subset of efficient cleavage sites. J Interferon Cytokine Res 24: 664-676.
Hassel B A, Zhou A, Sotomayor C, Maran A, Silverman R H. 1993. A dominant negative mutant of 2-5A-dependent RNase suppresses antiproliferative and antiviral effects of interferon. EMBO J 12: 3297-3304.
Horner S M, Gale M Jr. 2009. Intracellular innate immune cascades and interferon defenses that control hepatitis C virus. J Interferon Cytokine Res 29: 489-498.
Hovanessian A G, Justesen J. 2007. The human 2′-5′oligoadenylate synthetase family: Unique interferon-inducible enzymes catalyzing 2′-5′ instead of 3′-5′ phosphodiester bond formation. Biochimie 89: 779-788.
Hovanessian A G, Brown R E, Kerr I M. 1977. Synthesis of low molecular weight inhibitor of protein synthesis with enzyme from interferon-treated cells. Nature 268: 537-540.
Kato H, Sato S, Yoneyama M, Yamamoto M, Uematsu S, Matsui K, Tsujimura T, Takeda K, Fujita T, Takeuchi O, et al. 2005. Cell type-specific involvement of RIG-I in antiviral response. Immunity 23: 19-28.
Kato H, Takeuchi O, Sato S, Yoneyama M, Yamamoto M, Matsui K, Uematsu S, Jung A, Kawai T, Ishii K J, et al. 2006. Differential roles of MDA5 and RIG-I helicases in the recognition of RNA viruses. Nature 441: 101-105.
Kawai T, Takahashi K, Sato S, Coban C, Kumar H, Kato H, Ishii K J, Takeuchi O, Akira S. 2005. IPS-1, an adaptor triggering RIG-I- and Mda5-mediated type I interferon induction. Nat Immunol 6: 981-988.
Kerr I M, Brown R E. 1978. pppA2′p5′A2′p5′A: An inhibitor of protein synthesis synthesized with an enzyme fraction from interferon-treated cells. Proc Natl Acad Sci 75: 256-260.
Kumar H, Kawai T, Kato H, Sato S, Takahashi K, Coban C, Yamamoto M, Uematsu S, Ishii K J, Takeuchi O, et al. 2006. Essential role of IPS-1 in innate immune responses against RNA viruses. J Exp Med 203: 1795-1803.
Lee H, Shin H, Wimmer E, Paul A V. 2004. cis-acting RNA signals in the NS5B C-terminal coding sequence of the hepatitis C virus genome. J Virol 78: 10865-10877.
Loo Y M, Owen D M, Li K, Erickson A K, Johnson C L, Fish P M, Carney D S, Wang T, Ishida H, Yoneyama M, et al. 2006. Viral and therapeutic control of IFN-β promoter stimulator 1 during hepatitis C virus infection. Proc Natl Acad Sci 103: 6001-6006.
Malathi K, Paranjape J M, Bulanova E, Shim M, Guenther-Johnson J M, Faber P W, Eling T E, Williams B R, Silverman R H. 2005. A transcriptional signaling pathway in the IFN system mediated by 2′-5′-oligoadenylate activation of RNase L. Proc Natl Acad Sci 102: 14533-14538.
Malathi K, Dong B, Gale M Jr, Silverman R H. 2007. Small self-RNA generated by RNase L amplifies antiviral innate immunity. Nature 448: 816-819.
Meylan E, Curran J, Hofmann K, Moradpour D, Binder M, Bartenschlager R, Tschopp J. 2005. Cardif is an adaptor protein in the RIG-I antiviral pathway and is targeted by hepatitis C virus. Nature 437: 1167-1172.
Palmenberg A C, Sgro J. 1997. Topological organization of picornaviral genomes: Statistical prediction of RNA structural signals. Seminars in Virology 8: 231-241.
Plumet S, Herschke F, Bourhis J M, Valentin H, Longhi S, Gerlier D. 2007. Cytosolic 5′-triphosphate ended viral leader transcript of measles virus as activator of the RIG I-mediated interferon response. PLoS ONE 2: e279. doi: 10.1371/journal.pone.0000279.
Rehwinkel J, Reis e Sousa C. 2010. RIGorous detection: Exposing virus through RNA sensing. Science 327: 284-286.
Saito T, Hirai R, Loo Y M, Owen D, Johnson C L, Sinha S C, Akira S, Fujita T, Gale M Jr. 2007. Regulation of innate antiviral defenses through a shared repressor domain in RIG-I and LGP2. Proc Natl Acad Sci 104: 582-587.
Saito T, Owen D M, Jiang F, Marcotrigiano J, Gale M Jr. 2008. Innate immunity induced by composition-dependent RIG-I recognition of hepatitis C virus RNA. Nature 454: 523-527.
Schlee M, Roth A, Hornung V, Hagmann C A, Wimmenauer V, Barchet W, Coch C, Janke M, Mihailovic A, Wardle G, et al. 2009. Recognition of 5′ triphosphate by RIG-I helicase requires short blunt double-stranded RNA as contained in panhandle of negative-strand virus. Immunity 31: 25-34.
Seth R B, Sun L, Ea C K, Chen Z J. 2005. Identification and characterization of MAVS, a mitochondrial antiviral signaling protein that activates NF-κB and IRF 3. Cell 122: 669-682.
Silverman R H. 2007. Viral encounters with OAS and RNase L during the IFN antiviral response. J Virol 81: 12720-12729.
Silverman R H, Skehel J J, James T C, Wreschner D H, Kerr I M. 1983. rRNA cleavage as an index of ppp(A2′p)nA activity in interferon-treated encephalomyocarditis virus-infected cells. J Virol 46: 1051-1055.
Sumpter R Jr, Loo Y M, Foy E, Li K, Yoneyama M, Fujita T, Lemon S M, Gale M Jr. 2005. Regulating intracellular antiviral defense and permissiveness to hepatitis C virus RNA replication through a cellular RNA helicase, RIG-I. J Virol 79: 2689-2699.
Taguchi T, Nagano-Fujii M, Akutsu M, Kadoya H, Ohgimoto S, Ishido S, Hotta H. 2004. Hepatitis C virus NS5A protein interacts with 2′,5′-oligoadenylate synthetase and inhibits antiviral activity of IFN in an IFN sensitivity-determining region-independent manner J Gen Virol 85: 959-969.
Takahasi K, Yoneyama M, Nishihori T, Hirai R, Kumeta H, Narita R, Gale M Jr, Inagaki F, Fujita T. 2008. Nonself RNA-sensing mechanism of RIG-I helicase and activation of antiviral immune responses. Mol Cell 29: 428-440.
Tanaka N, Nakanishi M, Kusakabe Y, Goto Y, Kitade Y, Nakamura K T. 2004. Structural basis for recognition of 2′,5′-linked oligoadenylates by human ribonuclease L. EMBO J 23: 3929-3938.
Thakur C S, Jha B K, Dong B, Das Gupta J, Silverman K M, Mao H, Sawai H, Nakamura A O, Banerjee A K, Gudkov A, et al. 2007. Small-molecule activators of RNase L with broad-spectrum antiviral activity. Proc Natl Acad Sci 104: 9585-9590.
Ting J P, Duncan J A, Lei Y. 2010. How the noninflammasome NLRs function in Tuplin A, Wood J, Evans D J, Patel A H, Simmonds P. 2002. Thermodynamic and phylogenetic prediction of RNA secondary structures in the coding region of hepatitis C virus. RNA 8: 824-841.
Tuplin A, Evans D J, Simmonds P. 2004. Detailed mapping of RNA secondary structures in core and NS5B-encoding region sequences of hepatitis C virus by RNase cleavage and novel bioinformatic prediction methods. J Gen Virol
Wakita T, Pietschmann T, Kato T, Date T, Miyamoto M, Zhao Z, Murthy K, Habermann A, Krausslich H G, Mizokami M, et al. 2005. Production of infectious hepatitis C virus in tissue culture from a cloned viral genome. Nat Med 11: 791-796.
Wreschner D H, McCauley J W, Skehel J J, Kerr I M. 1981. Interferon action-sequence specificity of the ppp(A2′p)nA-dependent ribonuclease. Nature
Xu L G, Wang Y Y, Han K J, Li L Y, Zhai Z, Shu H B. 2005. VISA is an adapter protein required for virus-triggered IFN-β signaling. Mol Cell 19: 727-740.
Yoneyama M, Fujita T. 2010. Recognition of viral nucleic acids in innate immunity. Rev Med Virol 20: 4-22.
Yoneyama M, Kikuchi M, Natsukawa T, Shinobu N, Imaizumi T, Miyagishi M, Taira K, Akira S, Fujita T. 2004. The RNA helicase RIG-I has an essential function in double-stranded RNA-induced innate antiviral responses. Nat Immunol 5: 730-737.
You S, Stump D D, Branch A D, Rice C M. 2004. A cis-acting replication element in the sequence encoding the NS5B RNA-dependent RNA polymerase is required for hepatitis C virus RNA replication. J Virol 78: 1352-1366.
Zhou A, Hassel B A, Silverman R H. 1993. Expression cloning of 2-5A-dependent RNAase: a uniquely regulated mediator of interferon action. Cell
Zhou A, Molinaro R J, Malathi K, Silverman R H. 2005. Mapping of the human RNASEL promoter and expression in cancer and normal cells. J Interferon Cytokine Res 25: 595-603.
Zuker M. 2003. Mfold web server for nucleic acid folding and hybridization prediction. Nucleic Acids Res 31: 3406-3415.

What is claimed is:

1. An isolated nucleic acid sequence comprising SEQ ID NO: 1.

2. The sequence of claim 1 further comprising one or more hydroxyl (—OH) groups, one or more monophosphoryl (-p) groups, one or more single stranded overhangs or a combination thereof.

3. The sequence of claim 2 further comprising a 5′-OH and a 3′-p.

4. The sequence of claim 2 further comprising a 5′-p₃ and a 3′-OH.

5. The sequence of claim 2 further comprising a 5′ single stranded overhang, a 3′ single stranded overhang or a combination thereof.

6. An isolated nucleic acid sequence comprising SEQ ID NO: 25.

7. An isolated nucleic acid sequence comprising SEQ ID NO: 26.

8. A pharmaceutical composition comprising the sequence of claim 1.

9. A pharmaceutical composition comprising the sequence of claim 6.

10. A pharmaceutical composition comprising the sequence of claim 7.

11. A method of inducing an immune response to a hepatitis C virus (HCV) in a cell comprising introducing into the cell a composition comprising a HCV svRNA; and maintaining the cell under conditions in which the svRNA stimulates RIG-1 signaling and propagates signaling to the IFN-β gene, thereby inducing an immune to the HCV in the cell.

12. The method of claim 11 wherein the composition comprises SEQ ID NO: 1, SEQ ID NO: 3, SEQ ID NO: 4, SEQ ID NO: 25, SEQ ID NO: 26, SEQ ID NO: 27 or a combination thereof.

13. The method of claim 11 wherein the cell is a mammalian cell.

14. The method of claim 13 wherein the mammalian cell is a human cell.

15. The method of claim 11 wherein the HCV is genotype 1a HCV.

16. A method of inducing an immune response to HCV in an individual in need thereof comprising administering a therapeutically effective amount of a composition comprising a HCV svRNA that stimulates RIG-1 signaling and propagates signaling to the IFN-β gene, thereby inducing an immune response to the HCV in the individual.

17. The method of claim 16 wherein the composition comprises SEQ ID NO: 1, SEQ ID NO: 3, SEQ ID NO: 4, SEQ ID NO: 25, SEQ ID NO: 26, SEQ ID NO: 27 or a combination thereof.

18. The method of claim 16 wherein the individual is a mammal.

19. The method of claim 18 wherein the mammal is a human.

20. The method of claim 16 wherein the HCV is genotype 1a HCV.

21. A method of treating a HCV in an individual in need thereof comprising administering a therapeutically effective amount of a composition comprising a HCV svRNA (e.g., HCV svRNA3) that stimulates RIG-1 signaling and propagates signaling to the IFN-β gene, thereby inducing an immune response to the HCV in the individual.

22. The method of claim 21 wherein the composition comprises SEQ ID NO: 1, SEQ ID NO: 3, SEQ ID NO: 4, SEQ ID NO: 25, SEQ ID NO: 26, SEQ ID NO: 27 or a combination thereof.

23. The method of claim 21 wherein the individual is a mammal.

24. The method of claim 23 wherein the mammal is a human.

25. The method of claim 21 wherein the HCV is genotype 1a HCV.


SEQUENCE LISTING (72 sequences)

SEQ ID NO: 1 - RNA, 130 nt, Unknown
  two adjacent stem-loops in the NS5B region of the HCV open reading frame
aguaagaacaaagcucaaacucacuccaauagcggccgcuggccggcuggacuuguccgguugguucacggcuggcuacagcgggggagacauuuaucacagcgugucucaugcccggccccgcugguuc

SEQ ID NO: 2 - RNA, 89 nt, Unknown
  svRNA3
agaacaaagcucaaacuccuccaauagcggccgcuggccggcuggacuuguccgguugguucacggcuggcuacagcgggggagacauu

SEQ ID NO: 3 - RNA, 93 nt, Unknown
  svRNA3 (5′-p3/3′-OH)
aagaacaaagcucaaacucacuccaauagcggccgcuggccggcuggacuuguccgguugguucacggcuggcuacagcgggggagacauuua

SEQ ID NO: 4 - RNA, 90 nt, Unknown
  svRNA3 (5′-OH/3′-p)
aagaacaaagcucaaacucacuccaauagcggccgcuggccggcuggacuuguccgguugguucacggcuggcuacagcgggggagacau

SEQ ID NO: 5 - RNA, 93 nt, Unknown
  complement of svRNA3 (5′p3/3′-OH) (CsvRNA3)
uaaaugucucccccgcuguagccagccgugaaccaaccggacaaguccagccggccagcggccgcuauuggagugaguuugagcuuuguucuu

SEQ ID NO: 6 - RNA, 47 nt, Unknown
  svRNA3 short (5′-OH 3′OH or 3′-p) (5′-OH/3′-OH or 3′p)
agcggccgcuggccggcuggacuuguccgguugguucacggcuggcu

SEQ ID NO: 7 - RNA, 77 nt, Unknown
  5′ delta svRNA3 (5′-p3/3′-OH)
cucacuccaauagcggccgcuggccggcuggacuuguccgguugguucacggcuggcuacagcgggggagacauuua

SEQ ID NO: 8 - RNA, 86 nt, Unknown
  3′ delta svRNA3 (5′-p3/3′-OH)
aagaacaaagcucaaacucacuccaauagcggccgcuggccggcuggacuuguccgguugguucacggcuggcuacagcgggggag

SEQ ID NO: 9 - RNA, 70 nt, Unknown
  5′3′ delta svRNA3 (5′-p3/3′-OH)
cucacuccaauagcggccgcuggccggcuggacuuguccgguugguucacggcuggcuacagcgggggag

SEQ ID NO: 10 - DNA, 12 bp, Unknown
  svRNA1 for HCV nt 2413-2424
acctccaccaga

SEQ ID NO: 11 - DNA, 14 bp, Unknown
  svRNA2 for HCV nt 4122-4135
acgctgggctttgg

SEQ ID NO: 12 - DNA, 18 bp, Unknown
  svRNA3 for HCV nt 9204-9221
aaactcactccaatagcg

SEQ ID NO: 13 - DNA, 64 bp, Unknown
  svRNA4 for HCV nt 473-536
gggtgtgcgcgcgacgaggaagacttccgagcggtcgcaacctcgaggtagacgtcagcctatc

SEQ ID NO: 14 - DNA, 14 bp, Unknown
  svRNA5 for HCV nt 1228-1241
ggcgccactggacg

SEQ ID NO: 15 - DNA, 24 bp, Unknown
  svRNA6 for HCV nt 1381-1404
tgatcgctggtgctcactggggag

SEQ ID NO: 16 - DNA, 12 bp, Unknown
  svRNA7 for HCV nt 1645-1656
ccggctggttag

SEQ ID NO: 17 - DNA, 14 bp, Unknown
  svRNA8 for HCV nt 3403-3416
aggggtggaggttg

SEQ ID NO: 18 - DNA, 11 bp, Unknown
  svRNA9 for HCV nt 3429-3439
acggcgtacgc

SEQ ID NO: 19 - DNA, 17 bp, Unknown
  svRNA10 for HCV nt 3863-3879
acacgccgtgggcctat

SEQ ID NO: 20 - DNA, 13 bp, Unknown
  svRNA11 for HCV nt 5313-5324
agcacctgggtgc

SEQ ID NO: 21 - DNA, 13 bp, Unknown
  svRNA12 for HCV nt 7044-7056
caggagatgggcg

SEQ ID NO: 22 - DNA, 18 bp, Unknown
  svRNA13 for HCV nt 7308-7325
cctgtgcctccgcctcgg

SEQ ID NO: 23 - DNA, 14 bp, Unknown
  svRNA14 for HCV 8761-8775
accctacaaccccc

SEQ ID NO: 24 - DNA, 13 bp, Unknown
  svRNA15 for HCV nt 8822-8834
ctggctaggcaac

SEQ ID NO: 25 - RNA, 35 nt, Unknown
  svRNA1 for HCV nt 2395-2429
uguccaccggccucauccaccuccaccagaacauu

SEQ ID NO: 26 - RNA, 29 nt, Unknown
  svRNA2 for HCV nt 4112-4140
uguugcugcaacgcugggcuuuggugcuu

SEQ ID NO: 27 - RNA, 90 nt, Unknown; identical to SEQ ID NO: 4
  svRNA3 for HCV nt 9191-9281

SEQ ID NO: 28 - RNA, 65 nt, Unknown
  svRNA4 for HCV nt 472-536
ugggugugcgcgcgacgaggaagacuuccgagcggucgcaaccucgagguagacgucagccuauc

SEQ ID NO: 29 - RNA, 42 nt, Unknown
  svRNA5 for HCV nt 1215-1257
accuucucucccaggcgccacuggacgacgcaagacugcaau

SEQ ID NO: 30 - RNA, 142 nt, Unknown
  svRNA6 for HCV nt 1280-1423
acgggucaucgcauggcaugggauaugaugaugaacugguccccuacggcagcguuggugguagcucagcugcuccggaucccacaagccaucauggacaugaucgcuggugcucacuggggaguccuggcgggcauagcgu

SEQ ID NO: 31 - RNA, 214 nt, Unknown
  svRNA7 for HCV nt 1542-1755
ggucuccuuacaccaggcgccaagcagaacauccaacugaucaacaccaacggcaguuggcacaucaauagcacggccuugaauugcaaugaaagccuuaacaccggcugguuagcagggcucuucuaucaacacaaauucaacucuucaggcuguccugagagguuggccagcugccgacgccuuaccgauuuugcccagggcugggguccua

SEQ ID NO: 32 - RNA, 97 nt, Unknown
  svRNA8 for HCV nt 3374-3470
gcuugggccagccgacggaauggucuccaagggguggagguugcuggcgcccaucacggcguacgcccagcagacgagaggccuccuaggguguaua

SEQ ID NO: 33 - RNA, 97 nt, Unknown; identical to SEQ ID NO: 32
  svRNA9 for HCV nt 3374-3470

SEQ ID NO: 34 - RNA, 179 nt, Unknown
  svRNA10 for HCV nt 3733-3912
accuggucacgaggcacgccgaugucauucccgugcgccggcgaggugauagcagggguagccugcuuucgccccggcccauuuccuacuugaaaggcuccucgggggguccgcuguugugccccgcgggacacgccgugggccuauucagggccgcggugugcacccguggaguggcu

SEQ ID NO: 35 - RNA, 182 nt, Unknown
  svRNA11 for HCV nt 5218-5399
acagacugggcgcuguucagaaugaagucacccugacgcacccaaucaccaaauacaucaugacaugcaugucggccgaccuggagaucgucacgagcaccugggugcucguuggcggcguccuggcugcucuggccgcguauugccugucaacaggcugcguggucauagugggcaggauu

SEQ ID NO: 36 - RNA, 249 nt, Unknown
  svRNA12 for HCV nt 6984-7232
ugcaccgccaaccaugacuccccugacgccgagcucauagaggcuaaccuccuguggaggcaggagaugggcggcaacaucaccaggguugagucagagaacaaaguggugauucuggacuccuucgauccgcuuguggcagaggaggaugagcgggaggucuccguaccugcagaaauucugcggaagucucggagauucgcccgggcccugcccgucugggcgcggccggacuacaaccccccgcua

SEQ ID NO: 37 - RNA, 72 nt, Unknown
  svRNA13 for HCV nt 7289-7361
ccaccuccacgguccccuccugugccuccgccucggaaaaagcguacggugguccucaccgaaucaacccua

SEQ ID NO: 38 - RNA, 82 nt, Unknown
  svRNA14 for HCV nt 8754-8837
cccgugacccuacaaccccccucgcgagagccgcgugggagacagcaagacacacuccagucaauuccuggcuaggcaacau

SEQ ID NO: 39 - RNA, 82 nt, Unknown; identical to SEQ ID NO: 38
  svRNA15 for HCV nt 8754-8837

SEQ ID NO: 40 - DNA, 45 bp, Unknown
  HCV1up
gtaatacgactcactataggggccagccccctgatgggggcgaca

SEQ ID NO: 41 - DNA, 27 bp, Unknown
  HCV2318dn
cctgtccctgtcttccagatcacagcg

SEQ ID NO: 42 - DNA, 45 bp, Unknown
  HCV2316up
gtaatacgactcactatagggaggtccgagctcagcccattgctg

SEQ ID NO: 43 - DNA, 27 bp, Unknown
  HCV3948dn
cactaggttctccacagggataaagtc

SEQ ID NO: 44 - DNA, 45 bp, Unknown
  HCV3946up
gtaatacgactcactataggggagacaaccatgagatccccggtg

SEQ ID NO: 45 - DNA, 27 bp, Unknown
  HCV5435dn
cctgtcaggtataattgccggcttccc

SEQ ID NO: 46 - DNA, 45 bp, Unknown
  HCV5430up
gtaatacgactcactataggggacagggaggttctctaccaggag

SEQ ID NO: 47 - DNA, 24 bp, Unknown
  HCV6698dn
gggcgatgggatctggcacgggca

SEQ ID NO: 48 - DNA, 45 bp, Unknown
  HCV6696up
gtaatacgactcactatagggcccgaatttttcacagaattggac

SEQ ID NO: 49 - DNA, 24 bp, Unknown
  HCV8702dn
ggagcatgatgttataagctccaa

SEQ ID NO: 50 - DNA, 45 bp, Unknown
  HCV8703up
gtaatacgactcactatagggtccaacgtgtcagtcgcccacgac

SEQ ID NO: 51 - DNA, 24 bp, Unknown
  HCV9702dn
cgcggcataccccgcgtattccca

SEQ ID NO: 52 - DNA, 24 bp, Unknown
  HCV9416dn
ggaaatggcctaagaggccggagt

SEQ ID NO: 53 - DNA, 44 bp, Unknown
  svRNA3up
gtaatacgactcactatagggaagaacaaagctcaaactcactc

SEQ ID NO: 54 - DNA, 23 bp, Unknown
  svRNA3dn
taaatgtctcccccgctgtagcc

SEQ ID NO: 55 - DNA, 44 bp, Unknown
  csvRNA3up
gtaatacgactcactatagggtaaatgtctcccccgctgtagcc

SEQ ID NO: 56 - DNA, 24 bp, Unknown
  csvRNA3dn
aagaacaaagctcaaactcactcc

SEQ ID NO: 57 - DNA, 45 bp, Unknown
  polyU/UCup
gtaatacgactcactatagggaggccatttcctgttttttttttt

SEQ ID NO: 58 - DNA, 25 bp, Unknown
  polyU/UCdn
aaagaaggaaggaaaagaaaggaaa

SEQ ID NO: 59 - DNA, 44 bp, Unknown
  svRNA3 5ssdel up
gtaatacgactcactatagggctcactccaatagcggccgctgg

SEQ ID NO: 60 - DNA, 25 bp, Unknown
  svRNA3 3ssdel dn
ctcccccgctgtagccagccgtgaa

SEQ ID NO: 61 - DNA, 44 bp, Unknown
  svRNA4up
gtaatacgactcactataggggggtgtgcgcgcgacgaggaaga

SEQ ID NO: 62 - DNA, 24 bp, Unknown
  svRNA4dn
gataggctgacgtctacctcgagg

SEQ ID NO: 63 - DNA, 29 bp, Unknown
  mIFN-b probe
atggaaagatcaacctcacctacagggcg

SEQ ID NO: 64 - DNA, 26 bp, Unknown
  mRIG-1 probe
cacaaacttggagagtcacgggaccc

SEQ ID NO: 65 - DNA, 25 bp, Unknown
  mISG56 probe
tcccaactgaggacatcccgaaaca

SEQ ID NO: 66 - DNA, 22 bp, Unknown
  mIFN-b fwd
gaaaggacgaacattcggaaat

SEQ ID NO: 67 - DNA, 22 bp, Unknown
  mIFN-b rev
tccgtcatctccatagggatct

SEQ ID NO: 68 - DNA, 22 bp, Unknown
  mRIG-1fwd
gcaaaaaccacccatacaatca

SEQ ID NO: 69 - DNA, 20 bp, Unknown
  mRiG-1 rev
cgcggtcttagcatctccaa

SEQ ID NO: 70 - DNA, 21 bp, Unknown
  mISG56 fwd
agggctctgctacaagcaaca

SEQ ID NO: 71 - DNA, 21 bp, Unknown
  mISG56 rev
tgccaattcttgcacattgtc

SEQ ID NO: 72 - DNA, 21 bp, Unknown
  Ext up
aagaacaaagctcaactcact